(19) United States
(12) Patent Application Publication
(10) Pub. No.: US 2024/0351967 A1
(43) Pub. Date: Oct. 24, 2024

(54) METHODS OF PREPARATION OF ZINGERONE, COMPOSITIONS COMPRISING ZINGERONE, AND USES THEREFOR

(71) Applicant: Evithe Limited, Lower Hutt (NZ)

(72) Inventor: Cynthia HUNEFELD-GAIKEMA, Lower Hutt (NZ)

(73) Assignee: Evithe Limited, Lower Hutt (NZ)

(21) Appl. No.: 18/682,832

(22) PCT Filed: Aug. 11, 2022
(86) PCT No.: PCT/NZ2022/050102

§ 371 Date: Feb. 9, 2024

(30) Foreign Application Priority Data
Aug. 11, 2021 (NZ) 779010

(51) Int. Cl. C07C 41/36; A61K 31/12; A61K 31/351; A61K 36/9068; A61K 38/14; A61K 45/06

(52) U.S. Cl. CPC C07C 41/36; A61K 31/12; A61K 31/351; A61K 38/14; A61K 45/06; A61K 36/9068; A61K 2236/333

(57) ABSTRACT

Disclosed are methods for preparing zingerone from ginger using alkaline solutions, as well as compositions obtained using these methods, and methods for using these compositions. Specifically disclosed are pharmaceutical compositions, and treatment methods employing zingerone in combination with other therapeutics such as gentamicin, vancomycin, and cefotaxime.

### RELATED APPLICATION

This application claims the benefit of New Zealand provisional application number 779010 filed on 11 Aug. 2021, the entire contents of which are incorporated by reference herein.

### FIELD OF THE INVENTION

The present disclosure relates to methods for preparing zingerone and compositions comprising zingerone. Specifically noted are pharmaceutical compositions, and uses for these compositions.

### BACKGROUND OF THE INVENTION

Ginger (Zingiber officinale) is a flowering plant whose rhizome is widely used as a spice and in traditional medicine. If consumed in reasonable quantities, ginger has few negative side effects. It is on the FDA's “generally recognized as safe” list.

The characteristic fragrance and flavour of ginger result from volatile oils that compose 1-3% of the weight of fresh ginger, primarily consisting of zingerone, shogaols, and gingerols with [6]-gingerol (1-[4′-hydroxy-3′-methoxyphenyl]-5-hydroxy-3-decanone)

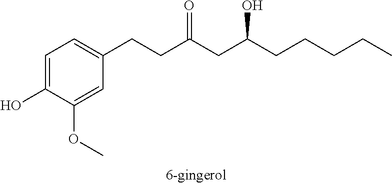 [SMILES: CCCCC[C@H](O)CC(=O)CCc1ccc(O)c(OC)c1]

as the major pungent compound.

Zingerone (also known as gingerone) has been reported as being produced from gingerols during drying or heat treatment at temperatures of about 40 degrees Celsius as reported by Li et. al., 2016, “Chemical Characterisation and antioxidant activities comparison in fresh, dried, stir frying and carbonized ginger” Journal of Chromatography B Analyt. Technol. Biomed. Life Sci. 1011:223-232. Zingerone has a lower pungency and a spicy-sweet aroma. Zingerone is also called vanillylacetone and is a crystalline solid that is reported to be sparingly soluble in water and soluble in ether. Zingerone's water solubility value of 0.57 g/L and Log P value of 2.02 1.92 and log S of −2.5 is described on the FoodB compounds database; see https://foodb.ca/compounds/FDB010527.

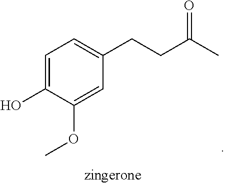

Fresh ginger contains minimal zingerone, and it is known to be produced by cooking or drying of the ginger root, which causes dehydration of gingerol through the loss of a water molecule to produce zingerone and hexanal. See, e.g., Gopi et al. 2016, “Study on temperature dependent conversion of active components of ginger” Int. J. of Pharma Sciences 6 (1): 1344-1347.

Shogaols are more pungent and have higher antioxidant activity and are not found in raw ginger, but are formed from gingerols during heating, storage or via acidity.

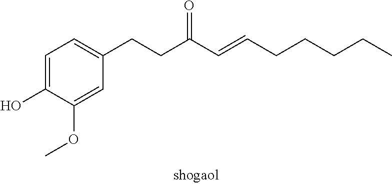 [SMILES: CCCCC/C=C/C(=O)CCc1ccc(O)c(OC)c1]

Shogaol is a dehydrated form of gingerol.

Zingerone was first isolated from the ginger root in 1917 by Hiroshi Nomura. Nomura identified and later patented (U.S. Pat. No. 1,263,796, issued Apr. 23, 1918) a method for the synthesis of zingerone, in which vanillin and acetone are reacted under basic conditions to form dehydrozingerone. This compound was obtained in about 95% quantity. This reaction was followed by catalytic hydrogenation of the intermediate compound in order to form zingerone, obtained in approximately 100% quantity.

Ginger compounds have been shown to be active against enterotoxigenic Escherichia coli heat-labile enterotoxin-induced diarrhoea. This type of diarrhoea is the leading cause of infant death in developing countries. It has been reported that Zingerone is likely the active constituent responsible for the antidiarrheal efficacy of ginger. The study concluded that ginger's bioactive compounds significantly blocked the binding of enterotoxigenic Escherichia coli heat-labile enterotoxin to cell-surface receptor G M1, resulting in the inhibition of fluid accumulation in the closed ileal loops of mice. See, e.g., Chen et al., 2007, “Ginger and its bioactive component inhibit enterotoxigenic Escherichia coli heat-labile enterotoxin-induced diarrhoea in mice” Journal of Agricultural and Food Chemistry 55 (21): 8390-7.

Zingerone has been shown to have an anti-inflammatory effect on liver inflammation in a peritonitis mouse model as reported by Kumar et al. See Kumar et al., “Zingerone suppresses liver inflammation induced by antibiotic mediated endotoxemia through down regulating hepatic mRNA expression of inflammatory markers in Pseudomonas aeruginosa peritonitis mouse model” PLOS ONE 9 (9): e106536.

Kumar et al. have also reported that zingerone can enhance the susceptibility of Pseudomonas aeruginosa cells to antibiotics. See Kumar et al., 2014, Life Sciences 117:24-32. Kumar et al. concluded that zingerone was found to cause alterations in the cell surface properties of Pseudomonas aeruginosa thereby increasing the susceptibility of Pseudomonas aeruginosa cells to antibiotics.

Limited research has been performed on optimising the production of zingerone from natural sources. Given the current emphasis on compositions derived from natural sources, there is a need for new compositions, including plant-based compositions, and particularly those with anti-microbial activity. The present application aims to meet these and other needs.

### SUMMARY

In one aspect, this disclosure encompasses a method of producing zingerone by: (i) subjecting ginger root to an alkaline treatment in alkaline solution; or (ii) subjecting juice obtained from ginger root to an alkaline treatment in an alkaline solution.

In specific aspects:

The ginger root is fresh.

The ginger root is dried.

The ginger root is dried at about 40 to about 70 degrees Celsius, or at about 55 to about 65 degrees Celsius, or at about 60 degrees Celsius.

The juice is obtained by macerating and/or pressing the ginger root.

The ginger root is diced and subjected to the alkaline treatment.

The ginger root is diced, dried and subjected to the alkaline treatment.

The alkaline treatment is carried out at about 40 to about 70 degrees Celsius.

The alkaline treatment is carried out at about 50 to about 60 degrees Celsius.

The alkaline treatment is carried out at about 55 to about 65 degrees Celsius.

The alkaline treatment is carried out at about 60 degrees Celsius.

The alkaline treatment is carried out for about 1-72 hours.

The alkaline treatment is carried out for about 1-48 hours.

The alkaline treatment is carried out for about 1-24 hours.

The alkaline treatment is carried out for about 1-30 hours, or about 1-20 hours, or about 1-10 hours, or about 1-5 hours.

The alkaline treatment is carried out for about 0.5 to about 3 hours, or about 0.5 to about 2 hours, or about 1 to about 2 hours.

The alkaline treatment is carried out for about 2 hours.

The alkaline treatment is carried out for about 1 hour.

Potassium hydroxide (KOH) is used.

A liquid form of potassium hydroxide (KOH) is used.

About 0.1% to about 1.0% KOH (v/v) is used. About 0.5% to about 0.7% KOH (v/v) is used.

About 1% to about 3% KOH (v/v) is used. About 1.5% to about 2.5% KOH (v/v) is used. About 2% KOH (v/v) is used.

Calcium hydroxide Ca(OH)₂ is used.

About 0.5% to about 4% Ca(OH)₂ (v/v) is used. About 1.5% to about 3.5% Ca(OH)₂ (v/v) is used. About 2% to about 3% Ca(OH)₂ (v/v) is used.

After alkaline treatment, the alkaline solution is neutralised.

The alkaline solution is neutralised to obtain a pH of about 6.5 to about 7.5.

The neutralised alkaline solution is freeze dried.

The neutralised alkaline solution is subjected to extraction of the zingerone.

The neutralised alkaline solution is dried and then subjected to extraction of the zingerone.

The drying is at about 60 degrees Celsius for up to 24 hours.

The zingerone is optionally extracted by one or more alcohol extraction steps. The zingerone is extracted by one or more ethanol extraction steps.

The ethanol extraction is carried out for at least 7 days.

The ethanol extraction is carried out for 24 hours or less.

The zingerone is extracted using supercritical fluid extraction.

The zingerone is extracted by a supercritical fluid extraction followed by an alcohol extraction step.

The method produces a product that consists essentially of zingerone.

The method produces a product that is free from or substantially free from aldehydes.

In one aspect, this disclosure encompasses a method of producing zingerone by subjecting ginger root extract to an alkaline treatment.

The ginger root extract is obtained by supercritical fluid extraction of the ginger root.

The ginger root extract is obtained by alcohol extraction of the ginger root.

The ginger root extract is obtained by juicing the ginger root.

The juicing includes macerating and/or pressing the ginger root.

The alkaline treatment is carried out at about 30 to about 70 degrees Celsius.

The alkaline treatment is carried out at about 50 about 60 degrees Celsius.

The alkaline treatment is carried out at about 55 to about 65 degrees Celsius.

The alkaline treatment is carried out at about 60 degrees Celsius.

The alkaline treatment is carried out for about 1-72 hours.

The alkaline treatment is carried out for about 1-48 hours.

The alkaline treatment is carried out for about 1-24 hours.

The alkaline treatment is carried out for about 1-30 hours, or about 1-20 hours, or about 1-10 hours, or about 1-5 hours.

The alkaline treatment is carried out for about 0.5 to about 3 hours, or about 0.5 to about 2 hours, or about 1 to about 2 hours.

The alkaline treatment is carried out for about 2 hours.

The alkaline treatment is carried out for about 1 hour.

Potassium hydroxide (KOH) is used.

A liquid form of potassium hydroxide (KOH) is used.

About 0.1% to about 5.0% KOH (v/v) is used. About 0.5% to about 0.7% KOH (v/v) is used.

About 1% to about 3% KOH (v/v) is used. About 1.5% to about 2.5% KOH (v/v) is used. About 2% KOH (v/v) is used.

Calcium hydroxide Ca(OH)₂ is used.

About 0.5% to about 4% Ca(OH)₂ (v/v) is used. About 1.5% to about 3.5% Ca(OH)₂ (v/v) is used. About 2% to about 3% Ca(OH)₂ (v/v) is used.

After alkaline treatment, the alkaline solution is neutralised.

The alkaline solution is neutralised to obtain a pH of about 6.5 to about 7.5.

After neutralisation of the alkaline solution, the zingerone is further extracted.

The zingerone is optionally further extracted by one or more alcohol extraction steps.

The zingerone is optionally further extracted by one or more ethanol extraction steps.

The ethanol extraction is carried out for at least 7 days.

The ethanol extraction is carried out for 24 hours or less.

The zingerone is optionally further extracted using supercritical fluid extraction.

The zingerone is optionally further extracted by a supercritical fluid extraction followed by an alcohol extraction step.

The method produces a product that consists essentially of zingerone.

The method produces a product that is free from or substantially free from aldehydes.

The method comprises (i) subjecting ginger root to an alkaline treatment in alkaline solution; or (ii) subjecting juice obtained from ginger root to an alkaline treatment in an alkaline solution, wherein the alkaline solution comprises about 1.5% to about 2.5% KOH (v/v), wherein the alkaline treatment is carried out for about 1 to about 2 hours, and wherein following alkaline treatment, the alkaline solution is neutralised to a pH of about 6.5 to about 7.5.

Also encompassed is a composition comprising zingerone, the zingerone being prepared by a method of any one of the preceding aspects.

Further encompassed is a composition consisting essentially of zingerone, the zingerone being prepared by a method of any one of the preceding aspects.

In one aspect, this disclosure encompasses a method of treating or preventing an infection by a microbial organism, comprising: administering to a subject a composition of any one of the preceding aspects, thereby treating or preventing the infection.

In one other aspect, this disclosure encompasses the use of a composition of any one of the preceding aspects for preparing a medicament for treating or preventing an infection by a microbial organism.

Also encompassed is a composition comprising zingerone, for treating or preventing an infection by a microbial organism.

### In Various Aspects:

The composition comprising zingerone is obtained by a method of any one of the preceding aspects.

The composition comprising zingerone is obtained from a ginger root.

The composition comprising zingerone is obtained from fresh ginger root.

The composition comprising zingerone is obtained from dried ginger root.

The composition comprising zingerone is obtained from juice prepared from a ginger root.

The juice is prepared by macerating and/or pressing of the ginger root.

The composition comprising zingerone is obtained using an alkaline conversion step to convert the gingerol in the ginger root, or in the juice from ginger root, to zingerone.

The composition comprising zingerone is free from or substantially free from aldehydes.

The composition comprising zingerone is formulated as a powder.

The composition comprising zingerone is formulated as a tincture.

The composition further comprises one or more anti-microbial agents.

The composition further comprises one or more aminoglycoside antibiotic agents and/or one or more glycopeptide antibiotic agents.

The composition further comprises vancomycin.

The composition further comprises gentamicin.

The microbial organism is selected from the group consisting of: bacteria, and fungi.

The bacteria are a Gram positive bacteria or a Gram negative bacteria.

The bacteria are selected from the group consisting of: Bacillus, Clostridium, Escherichia, Mycoplasma, Neissaria, Pseudomonas, Salmonella, Shigella, Streptococcus, Staphylococcus, and Vibrio bacteria.

The bacteria are selected from the group consisting of: Escherichia coli, Staphylococcus aureus, Streptococcus pneumoniae, Pseudomonas aeruginosa, and Klebsiella pneumoniae.

The fungi are selected from the group consisting of: Aspergillus, Candida, Coccidioides, Cryptococcus, Histoplasma, Pneumocystis, and Stachybotrys fungi.

The fungi are selected from the group consisting of: Candida albicans, Aspergillus species, Histoplasma capsulatum, Coccidioides immitis, and Pneumocystis carinii, and tinea fungi.

In various aspects for the composition:

The composition is formulated for topical administration, or oral administration.

The composition is formulated as a liquid, powder, tablet or capsule.

The composition comprises a dose of about 1 mg to about 5000 mg of zingerone

The composition comprises a dose of about 1 mg to about 1500 mg of zingerone

The composition comprises a dose of about 5 mg to about 500 mg of zingerone.

The composition comprises a dose of about 1 mg to about 150 mg of zingerone.

The composition comprises a dose of about 5 mg to about 50 mg of zingerone.

The composition comprises a dose of about 1 mg to about 15 mg of zingerone.

The composition comprises a dose of about 1 mg to about 10 mg of zingerone.

The composition is provided in a sachet.

The composition is formulated for co-administration with one or more anti-microbial agents.

The composition is formulated for co-administration with one or more aminoglycoside antibiotic agents and/or one or more glycopeptide antibiotic agents.

The composition is formulated for co-administration with gentamicin or vancomycin.

The microbial infection is an infection affecting one or more of: skin, eye, ear, nose, mouth, throat, oesophagus, lung, circulatory system, gastrointestinal system, or genitourinary system.

The foregoing brief summary broadly describes the features and technical advantages of certain embodiments of this disclosure. Further technical advantages will be described in the detailed description and examples that follows.

Novel features that are believed to be characteristic will be better understood from the detailed description when considered in connection with any accompanying figures and examples. However, the figures and examples provided herein are intended to help illustrate what is disclosed or assist with developing an understanding what is disclosed, and are not intended to limit the scope of this disclosure.

### BRIEF DESCRIPTION OF THE DRAWINGS

FIG. 1. Photograph depicting fresh ginger root.

FIG. 2. HPLC UV chromatogram traces (280 nm) for alkaline treated ginger.

FIG. 3. Schematic showing processing comparison.

FIG. 4A: Photograph depicting juicing machine and raw ginger prior to juicing.

FIG. 4B: Photograph depicting juicing of raw ginger in progress.

FIG. 5A: Ginger juice treated with KOH (0.5%) and analysed by HPLC.

Peak areas shown for zingerone (Z) and gingerol (G).

FIG. 5B: Ginger juice treated with KOH (1%) and analysed by HPLC.

Peak areas shown for zingerone (Z) and gingerol (G).

FIG. 5C: Ginger juice treated with KOH (2%) and analysed by HPLC.

Peak areas shown for zingerone (Z) and gingerol (G).

FIG. 6A: Ginger marc produced by pressing.

FIG. 6B: Ginger juice produced by pressing

FIG. 7: Schematic showing ethanolic extraction process and evaporation.

FIG. 8A: GCMS TIC analysis for ethanolic extract.

FIG. 8B: Comparison of ethanolic extract with hexanal standard. Shown are 2-7 minute regions of chromatogram.

FIG. 9A: Chequerboard assay for the combination of zingerone and gentamicin.

FIG. 9B: Chequerboard assay for the combination of zingerone and vancomycin.

FIG. 9C: Chequerboard assay for the combination of zingerone and cefotaxime.

### DETAILED DESCRIPTION

The following description sets forth numerous exemplary configurations, parameters, and the like. It should be recognised, however, that such description is not intended as a limitation on the scope of the present disclosure, but is instead provided as a description of exemplary embodiments.

All references, including patents and patent applications, cited in this specification are hereby incorporated by reference. No admission is made that any reference constitutes prior art. Nor does discussion of any reference constitute an admission that such reference forms part of the common general knowledge in the art, in New Zealand or in any other country.

### Definitions

In each instance herein, in descriptions, embodiments, and examples of the present disclosure, the terms “comprising”, “including”, etc., are to be read expansively, without limitation. Thus, unless the context clearly requires otherwise, throughout the description and the claims, the words “comprise”, “comprising”, and the like are to be construed in an inclusive sense as to opposed to an exclusive sense, that is to say in the sense of “including but not limited to”.

In the present description, the articles “a” and “an” are used to refer to one or to more than one (i.e., to at least one) of the grammatical object of the article. By way of example, “an element” can be taken to mean one element or more than one element.

Throughout this description, the term “about” is used to indicate that a value includes the standard deviation of error for the method being employed to determine the value, for example, levels of compounds or dosage levels, as described in detail herein. In particular, the term “about” encompasses up to a 10% deviation (positive and negative) in the stated value or range.

The term “comprising”, as used herein, may refer to the presence of zingerone or a zingerone extract in a composition. As exemplifications, the zingerone or zingerone extract may be at least 0.1%, at least 0.2%, at least 0.3%, at least 0.4%, at least 0.5%, at least 0.6%, at least 0.7%, at least 0.8%, at least 0.9%, at least 1%, at least 2%, at least 5%, at least 10%, at least 20%, at least 30%, at least 40%, at least 50%, at least 60%, at least 70%, at least 80%, or at least 90 by weight of the composition (% w/w). For liquids, the zingerone or zingerone extract may be at least 0.1%, at least 0.2%, at least 0.3%, at least 0.4%, at least 0.5%, at least 0.6%, at least 0.7%, at least 0.8%, at least 0.9%, at least 1%, at least 2%, at least 5%, at least 10%, at least 20%, at least 30%, at least 40%, at least 50%, at least 60%, at least 70%, at least 80%, or at least 90 by volume of the composition (% v/v).

The term “consisting essentially of”, as used herein, may refer to the presence of zingerone in a product. The product may be, for example, a composition as described herein, or may be, for example, a product produced by a method as described herein. As exemplifications, the zingerone may be at least 90% by weight of the product, or at least 91%, at least 92%, at least 93%, at least 94%, at least 95%, at least 96%, at least 97, at least 98%, at least 99%, by weight of the product (% w/w). For liquids, the zingerone may be at least 90% by volume of the product, or at least 91%, at least 92%, at least 93%, at least 94%, at least 95%, at least 96%, at least 97%, at least 98%, at least 99%, at least 99.5%, at least 99.8%, or at least 99.9% by volume of the product (% v/v).

The term “substantially free” in relation to aldehydes refers to a product having negligible aldehyde levels. The product may be, for example, a composition as described herein, or may be, for example, a product produced by a method as described herein. As exemplifications, aldehyde levels may be less than 20 ppm, less than 15 ppm, less than 10 ppm, less than 7.5 ppm, less than 5 ppm, less than 2 ppm, less than 1.5 ppm, less than 1 ppm, less than 0.75 ppm, less than 0.5 ppm, less than 0.2 ppm, less than 0.1 ppm, less than 0.05 ppm, less than 0.005 ppm, or less than 0.0005 ppm.

The term “alkaline treatment” as used herein means the exposure of a sample of ginger or ginger extract to an aqueous solution containing alkali having a pH greater than 7. Included, without limitation, are solutions comprising, sodium hydroxide, potassium hydroxide, calcium hydroxide, magnesium hydroxide and any combination thereof. It is to be appreciated that the alkaline treatment can occur at a range of temperatures as described herein and that the alkaline solution may be heated prior to or during exposure to the sample comprising ginger or an extract from ginger. The alkaline solution will have a chemically effective amount of alkali present to convert at least some gingerol present in the sample to zingerone. Particular methodologies are described in detail herein.

An “extract”, as prepared from ginger root, refers to a composition where one or more liquid, solid, or chemical constituents of the root has been isolated or concentrated. For example, a liquid, solid, or semi-solid extract may be obtained from ginger root. An extract may be obtained by one or more of: juicing, pressing, macerating, mashing, milling, or other known processes. Solvent-based extraction is also included. Solid extracts are specifically noted, for example, powders obtained from drying or evaporation. As specific exemplifications, an extract may be prepared as a dry form, or may be prepared in the form of a solution. A “zingerone extract” refers to an extract comprising zingerone, or consisting essentially of zingerone, as prepared/produced from ginger root. Particular extracts and their production methods are described in detail herein.

A “pharmaceutical composition” refers to a composition administered to a subject, for example, to treat or prevent an infection.

“Microbes” or “microbial organisms”, as used herein, includes pathogenic organisms, such as bacteria, fungi, protozoa, and viruses. This includes all deleterious microbial organisms, including organisms associated with various infections and other health conditions, such as human and non-human diseases.

“Bacteria”, as used herein, includes but is not limited to: Bacillus, Bartonella, Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio, and Yersinia bacteria. Gram positive and Gram negative bacteria are included. Particular bacterial organisms are described in detail herein.

“Fungi” as used herein, includes but is not limited to: Aspergillus, Candida, Cryptococcus, Histoplasma, Pneumocystis, and Stachybotrys fungi. Tinea fungi are specifically included. Particular fungal organisms are described in detail herein.

“Protozoa” as used herein, includes but is not limited to: Acanthamoeba, Cryptosporidium, Entamoeba, Giardia, Leishmania, Plasmodium, Trypanosoma, and Toxoplasma protozoa. Particular protozoan organisms are described in detail herein.

An “anti-microbial agent” refers to a constituent that deters microbial growth or infections, e.g., medicines, herbal compositions, essential oils, and various other constituents to assist in the inhibition or elimination of microbes. Such may be utilised in combination with the compounds and extracts of this disclosure.

As used herein, a “subject” may be a human or non-human animal, particularly a mammal, including cattle, sheep, goats, pigs, horses, and other livestock, including, as well, dogs, cats, and other domesticated pets. In particular aspects, the subject is a human being.

“Treating” as used herein is meant as reducing or resolving an infection, or remedying a disease or other condition caused by an infection. It is expected that a treatment will result in the reduction in/or elimination of an infection. A treatment may halt, or reduce, or slow the progression of an infection.

“Preventing” as used herein is meant as impeding the onset of an infection, or the onset of a disease or other condition resulting from an infection. It is expected that a preventative measure will halt or delay an infection from occurring, or will reduce the severity of an infection if such should arise. It should be understood that the term “treating or preventing” does not exclude the possibility of obtaining both treatment and prevention (e.g., at the same time or at different times) of a disorder in any given subject.

“Inhibiting” or “halting” growth, as used herein, refers to a slowing or a stopping in growth of one or more microbial organisms. This can be achieved by killing the one or more microbial organisms, e.g., eradicating or reducing the microbial organisms that are present, or eradicating or reducing the reproductive units of the microbe(s), e.g., spores. Alternatively, or in addition to this, inhibiting or halting of growth can be achieved by partly or fully preventing the division or replication of the microbial organisms.

### Methods of Preparing Compositions

The inventors have found that zingerone compositions prepared from ginger root in accordance with the disclosed methods have significant anti-microbial activity. Therefore, the present disclosure relates generally to a zingerone composition prepared from ginger root, and methods of preparation of such.

In one aspect, this disclosure provides a method of producing zingerone from ginger root by subjecting the ginger root to an alkaline treatment. The alkaline treatment may include incubation in an alkaline solution as described herein. As starting material, the ginger root may be fresh ginger root. For example, to assist with preparation, it may be helpful to optimise the period of time that the ginger root is retained in the soil prior to harvesting. In this way, the ginger root that is utilised will be fresh and will retain the advantageous characteristics of fresh ginger root.

As exemplifications, to optimise freshness, the ginger root may be harvested less than 48 hours before processing, less than 24 hours before processing, or less than 12 hours before processing, or less than 6 hours before processing, or less than 3 hours before processing. For example, fresh ginger may have a moisture content of about 80% to about 95%, about 81% to about 95%, or about 82% to about 95%, or about 83% to about 95%, or about 85% to about 95% on a wet basis.

Alternatively, the ginger root may be dried prior to treatment. For example, The ginger root may be dried at about 40° to about 70° C., or at about 55° to about 65° C., or at about 60° C. Drying may be carried out for about 1-72 hours, or about 1-48 hours, or about 1-24 hours, or about 1-20 hours, or about 1-18 hours, or about 1-10 hours, or about 1-5 hours.

In certain aspects, the ginger root selected for use in the disclosed methods may have a minim level of gingerol, e.g., 6-gingerol. For example, the ginger root (e.g., fresh ginger root) may have about 0.3 to about 10 mg/g, or about 0.3 to about 9 mg/g, or about 0.3 to about 8 mg/g, or about 0.3 to about 7 mg/g, or about 0.3 to about 6 mg/g, or about 0.4 to about 5 mg/g of 6-gingerol. As further exemplifications, the ginger root may have at least 1 mg/g, at least 2 mg/g, at least 3 mg/g, at least 4 mg/g, or at least 5 mg/g of 6-gingerol. Thus, in certain circumstances, it may be advantageous to test levels of gingerol, e.g., 6-gingerol, in the starting material before commencing a method as disclosed herein.

As one aspect, the method comprises subjecting juice from the ginger root to an alkaline treatment. The ginger juice may be obtained by macerating and/or pressing. The macerating may comprise homogenising with a blender, food processor, or similar machinery. For pressing, machine or hand presses may be utilised. Screw pressing is specifically noted. The solid material remaining after juicing (ginger marc) may be re-juiced to obtain ginger juice. This may be repeated as needed. The various juice samples may be combined before alkaline treatment.

Optionally, the ginger marc may be subjected to hot water treatment to obtain a diluted juice. For example, water may be added to the marc at a ratio of about 6 to about 1 (˜6:1), or about 5 to about 1 (˜5:1), or about 4 about 1 (˜4:1), or about 3 to about 1 (˜3:1) by weight. The water may be, for example, at about 40° C. to about 80° C., or at about 50° C. to about 70° C., or at about 55° C. to about 65° C., or at about 60° C. The incubation time in the water may be about 5 minutes to about 60 minutes, or about 10 minutes to about 30 minutes, or about 15 minutes to about 20 minutes, or about 15 minutes. The diluted juice samples may then be subjected to alkaline treatment. The diluted juice samples may be combined with other juice samples before alkaline treatment.

In one aspect, potassium hydroxide (KOH) may be used in the alkaline treatment. For example, a liquid form of KOH may be used in the alkaline solution. The concentration of KOH used in the treatment mixture may be, for example, about 0.1% to about 1.0%, or about 0.5% to about 0.7%, or about 1% to about 3%, or about 1.5% to about 2.5%, or about 2% (v/v). As an alternative to this, calcium hydroxide Ca(OH)₂ may be used in the alkaline treatment. For example, a liquid form of Ca(OH)₂ may be used in the alkaline solution. The concentration of Ca(OH)₂ used in the treatment mixture may be, for example, about 0.5% to about 4%, about 1.5% to about 3.5%, about 1% to about 2%, or about 3.0% (v/v). Liquid forms may include, for example, stock solutions of about 25% to about 65%, or about 30% to about 60%, or about 35% to about 55%, or about 45% to about 55%, or about 50%.

In certain aspects, the alkaline treatment may achieve a pH level for the treatment solution of about pH 9 to about pH 14, or about pH 9.5 to about pH 13.5, or about pH 10 to about pH 13, or about pH 10.5 to about pH 12.5, or about pH 11 to about pH 12. The alkaline treatment may be carried out for a sufficient time and at a sufficiently elevated temperature to obtain desired levels of zingerone. For example, the alkaline treatment may be carried out for about 1-72 hours, or about 1-48 hours, or about 1-24 hours. Further examples include treatment for about 1 to about 30 hours, or about 1 to about 20 hours, or about 1 to about 10 hours, or about 1 to about 15 hours, or about 1 to about 7 hours, or about 1 to about 6 hours, or about 1 to about 5 hours, or about 1 to about 4 hours, or about 0.5 to about 3 hours, or about 0.5 to about 2 hours, or about 1 to about 2 hours, or about 2 hours, or about 1 hour. As particular examples, the alkaline treatment may be carried out at about 40° to about 70° C., or about 50° to about 60° C., or about 55° to about 65° C., or about 60° C. It will be understand that lower temperatures can allow for longer treatment periods. For example, alkaline treatments performed at room temperature can carried out for about 3 days to about 9 days, or for about 5 days to about 9 days, or for about 5 days to about 7 days.

Following alkaline treatment, the treatment mixture may be further processed, for example, by one or more of: neutralisation, extraction, and drying. For neutralisation, citric acid or other acid composition may be utilised. As exemplifications, neutralisation may achieve a pH of about 6.4 to about 7.4, or about 6.5 to about 7.5, or about 6.6 to about 7.6, or about 6.9 to about 7.4, or about 6.6, about 6.7, about 6.8, about 6.9, about 7.0, about 7.1, about 7.2, about 7.3, about 7.4, or about 7.5. For example, about 10 to about 700 g/L citric acid may be utilised, or about 10 to about 600 g/L, or about 10 to about 500 g/L, or about 10 to about 400 g/L, or about 10 to about 300 g/L, or about 10 to about 200 g/L, or about 10 to about 100 g/L, or about 10 to about 50 g/L, or about 10 to about 40 g/L, or about 10 to about 30 g/L, or about 10 to about 20 g/L, or about 15 to about 16 g/L citric acid may be utilised.

For extraction, zingerone extraction may be achieved by one or more alcohol extractions, e.g., one or more ethanol extractions. As exemplifications, an alcohol extraction, e.g., ethanol extraction, may be carried out for about 1-72 hours, or about 1-48 hours, or about 1-24 hours, or about 6-24 hours, or about 8-24 hours, or about 12-24 hours, or about 18-24 hours. In one specific aspect, ethanol extraction at room temperature may be utilised for 24 hours or less. One or more drying steps may be used before and/or after extraction. For example, freeze drying may be utilised.

The composition may be prepared as an antiseptic, or pharmaceutical composition. The composition can also be prepared as a functional food or beverage, a natural ingredient (e.g., a natural additive), or a natural supplement (e.g., a dietary supplement). In various aspects, the composition may be prepared in liquid or solid form, or semi-solid form. Various formulations are encompassed in this disclosure. In certain aspects, it may be desirable to formulate the composition into a powder. The powder may be provided in free flowing form or as a solid cake. The composition may be provided as a powder for forming a suspension, powder for forming a solution, bulk granules, or bulk powder. The powder may be prepared as tablets or capsules, or other pharmaceutical formulations, as described in detail herein.

As part of the initial processing, the ginger root may be washed or sterilised. The plant component (e.g., fruit or seed) may be passed through an assembly having one or more roller brushes for removing any adhering foreign matter. Conventional washing techniques may then be employed. For example, it is possible to use a series of spray nozzles to wash the components. Wash additives aiding cleansing or reducing the bacteria count on the plant components may be employed according to local regulations and requirements. For example, the plant components may be washed by a chlorine wash and/or an ozone impregnated water wash followed by a fresh water rinse.

As noted, it may be desirable for a liquid or semi-solid zingerone composition to be prepared from ginger root. As described herein, zingerone components may be extracted by chemical means (e.g., solvent-based extraction). Solvent-based extraction may utilise one or more of: water, methanol, ethanol, or 2-propanol, extraction. Supercritical fluid extraction, for example CO₂ extraction may also be used to extract zingerone. Also suitable are emulsions, pastes, suspensions, and syrups. For example, in certain aspects, it may be desirable to use a paste from the ginger root or from the ginger root component (e.g., zingerone or zingerone extract). As an exemplification, the ginger root may be heated for several hours, strained, and reduced to a thick, concentrated form. Upon thickening, the paste can be spread on a flat sheet, or transferred to a packaging, for example, a bag, tube, jar, bottle, or other container. The paste may be transferred aseptically. It may be desired to prepare the paste from mature plant components. The paste may be a smooth preparation.

In specific aspects, the present disclosure encompasses mechanical means (e.g., juicing means such as maceration and/or pressing) for extracting zingerone from ginger root. In one embodiment, a pressing assembly may be adapted to perform a pulping or comminution process. Such process can be relatively mild and gentle (soft pulping) compared to conventional fruit pulping techniques. With soft pulping, no significant disintegration or lysis of cells is utilised. The press belts may be multiple loops rotated about a series of pulleys. The distance separating the press belts may decrease in the direction of travel of the plant component. In this way, increased force may be exerted upon the plant component as it travels along the length of the pressing assembly. In a particular aspect, a pressing assembly or mechanical press may be used to obtain juice from the ginger root, as described herein. Alternatively, or in addition to this, mechanical maceration may be used to obtain juice. For example, commercial juicing equipment may be utilised.

The ginger root component (e.g., zingerone or zingerone extract) may be processed by a freezing step. This may be followed by or used in conjunction with a drying or evaporation step. In an alternative embodiment, the component is dried or evaporated, and then processed to a powder without an intervening freezing step. Methods involving air drying or heat-assisted drying (e.g., oven drying) may be used. Drying may be obtained, for example, by one or more of: sun or solar drying, hot air drying, batch drying, rotary drying, tunnel drying, belt drying, fluidised bed drying, impingement drying, puff drying, drum drying, spray drying, vacuum drying, freeze drying, or osmotic drying. Exemplary temperatures for drying include about 50° C.-70° C., about 55° C.-65° C., or at least 50° C., at least 55° C., at least 60°, or at least 65° C. Evaporation may be obtained, for example, by one or more of: pan evaporation, batch evaporation, tube evaporation, rising film evaporation, falling film evaporation, rising-falling film evaporation, or agitated film evaporation. Combinations of various drying and evaporation methods may be used. For example, filtering followed by freeze drying may be used.

If freezing is used, it may be desirable to freeze the ginger root component (e.g., zingerone or zingerone extract) as soon as possible after it is produced to maintain freshness. However, freezing may be carried out within 24 or 48 hours, as needed. Freezing methodologies are well known by persons that are skilled in the art. Blast freezing is particularly desirable for use with the present disclosure. The component may be frozen in standard sized pales, which are used to collect the frozen product after processing. The component, for example, can be stored frozen (e.g., at −18° C.) until it is required. Optionally, the component may then be freeze dried, i.e., lyophilised. Freeze drying techniques are widely known and commonly used. The freeze-drying cycle may be up to 48 hours. In particular aspects, the process may be carried out to such that water formation is avoided, and the moisture content is minimised during processing. It will be understood that freeze drying/lyophilising does not exclude the use of higher temperatures (i.e., higher than freezing temperatures). For example, higher temperatures may be used for removing residual moisture during the secondary drying phase for lyophilisation/freeze drying procedures.

The resulting dried or evaporated component from ginger root (e.g., zingerone or zingerone extract) may then be milled into a powder, which can then be utilised as appropriate. Milling methods are well known and widely used by skilled persons in the art. Standard mesh sizes may be used to produce the powder, for example, US 20, US 23, US 30, US 35, US 40, US 45, or US 50 mesh sizes may be used. The sieve size for the powder may range from 1.0 to 0.3 mm; or 0.84 to 0.4 mm; or 0.71 to 0.5 mm; or may be about 1.0 mm, about 0.84 mm, about 0.71 mm, about 0.59 mm, about 0.5 mm, about 0.47 mm, about 0.465 mm, about 0.437 mm, about 0.4 mm, about 0.355 mm, or about 0.3 mm.

It will be understood that, for any liquid or semi-solid product obtained from ginger root, the liquid/semi-solid may be used in this form or may be dried or evaporated to obtain a powder form for use as an antiseptic, or pharmaceutical composition, as described herein. In the same way, it will be understood that, for any solid product obtained from ginger root, the solid may be used as such (e.g., with milling, sieving, or other processing), or may be re-suspended to obtain a liquid or semi-solid form for use as an antiseptic, or pharmaceutical composition, as described herein.

### Compositions

The inventor has found that zingerone compositions prepared from ginger root in accordance with the methods disclosed herein have significant anti-microbial properties that are useful for inhibiting or halting the growth of microbial organisms, as well as for treating or preventing infections from these organisms.

The composition of the present disclosure may be prepared as one or more of: an antiseptic composition, or a pharmaceutical composition. As non-limiting examples, the percentage of zingerone or zingerone extract in the composition may be about 0.01% to about 0.5%, or about 0.02% to about 0.2%, or about 0.03% to about 0.1%, or about 0.04% to about 0.09%, or about 0.05% to about 0.08%, or about 0.06% to about 0.07%, or a percentage of at least about 1%, at least about 5%, at least about 6%, at least about 10%, at least about 12%, at least about 15%, at least about 20%, at least about 23%, at least about 25%, or at least about 30%, or a percentage of about 6.25%, about 12.5%, or about 25%, these percentages being representative of v/v values for a liquid composition or w/w values or a solid or semi-solid composition.

For example, a solid or semi-solid composition may include about 1 to about 100 mg/g zingerone, or about 5 to about 50 mg/g zingerone, or about 5 to about 25 mg/g zingerone, or about 5 to about 20 mg/g zingerone, or about 5 to about 15 mg/g zingerone, or about 10 to about 15 mg/g zingerone, or about 10 to about 13 mg/g zingerone, or about 12 mg/g zingerone (w/w). Similarly, a liquid composition may include about 1 to about 100 mg/ml zingerone, or about 5 to about 50 mg/ml zingerone, or about 5 to about 25 mg/ml zingerone, or about 5 to about 20 mg/ml zingerone, or about 5 to about 15 mg/ml zingerone, or about 10 to about 15 mg/ml zingerone, or about 10 to about 13 mg/ml zingerone, or about 12 mg/ml zingerone (w/v).

In various aspects, antiseptic compositions may be prepared, for example: for use on hands (e.g., hand sanitizers), pre-operative tissue (e.g., surgical preparations for skin), mucous membranes (e.g., treatments for bladder, urethral, or vaginal infections, or cleansing of these cavities prior to medical procedures), wounds or burns (e.g., antiseptic ointments, bandages, or dressings), or mouth or throat (e.g., mouthwashes or antiseptic lozenges). Antiseptic formulations and their preparation are well known in the art. Sec, e.g., Antiseptic Prophylaxis and Therapy in Ocular Infections: Principles, Clinical Practice and Infection Control, 2002, Karger, Basel.

As non-limiting examples, antiseptic compositions may include one or more of: diluents (e.g., ethanol or other alcohol), emollients (e.g., PEG-45, palm kernel glycerides, or isopropyl myristate), humectants (e.g., glycerine or methylpropanediol), carriers (e.g., one or more oils), occlusive agents (e.g., mineral oil or dimethicone) other conditioning agents (e.g., behentrimonium methosulfate or polyquaternium-7), and surfactants (e.g., mild surfactants (e.g., amphoacetate, isethionate, sulfosuccinate, in particular, sodium lauroamphoacetate, sodium cocoyl isethionate, disodium oleoamido sulfosuccinate, sodium lauryl sulfate, sodium C14-16 olefin sulfonate). Exemplary oils include olive oil, coconut oil (e.g., coconut-derived MCT oil), palm oil (e.g., palm kernel-derived MCT oil), any other MCT oil (medium-chain triglyceride oil), and any combination thereof. Other possible carriers include lecithin (e.g., liquid form) and propylene glycol. Any combination of the carriers set out herein is also noted.

In yet other aspects, pharmaceutical compositions may be prepared for various routes of administration, including topical or oral formulas. Also included are compositions prepared for other routes of enteral or parenteral administration. Enteral formulations include but are not limited to: oral, rectal, sublingual, sublabial, and buccal preparations. Parenteral formulations include but are not limited to: nasal, intraocular, vaginal, intralesional, transdermal, and transmucosal preparations. Methods for formulating pharmaceutical compositions are well known in the art. Sec, e.g., Remington: Essentials of Pharmaceutics, 2012, Pharmaceutical Press, London.

In particular aspects, the composition of this disclosure may be prepared as a powder, or in any other suitable dosage form. Topical formulations may be prepared, for example, as aerosols, balms, creams, dressings, drops, emulsions, films, foams, gels, liquids, lotions, masks, oils, ointments, pastes, powders, salves, soaps, sprays, suspensions, solutions, tinctures, and vapours. Further topical formulations include bandages, dressings, patches, pads, sponges, strips, tapes, and others noted herein.

As described herein, the composition may be formulated as a semi-solid or liquid composition, for example, for oral administration, or as a solution for enteral or parenteral administration. Alternatively, the composition may be formulated as a powder to be encapsulated, tableted, or added to or incorporated in other products.

Oral formulations may be prepared, for example, as draughts, drops, elixirs, emulsions, liquids, linctuses, solutions, sprays, suspensions, syrups, tonics, or, as films, gels, gummies, jellies, lozenges, nuggets, pastes, purees, pomaces, powders, pills, or strips. In other aspects, oral formulations may be prepared as a tablets or as capsules, for example, with liquid, semi-solid, or solid contents. Oral formulations may be provided in sachet form, for example, a powder sachet, or a gel or jelly sachet. Included also are oral formulations comprising thin strips, or comprising solids in a capsule to mix with food or drink. The oral formulation may be provided as shooters or shots (to be consumed by mouth), for example, liquid shots, gel or jelly shots, paste shots, or powder shots.

Particularly encompassed are delayed release formulas, extended release formulas, as well as formulas for rapid disintegration. Capsules, for example gel capsules, are specifically encompassed, as well as sachets and chewable tablets. Additionally, included are combination formulas, which include the powder of the present disclosure mixed with other beneficial agents, e.g., one or more anti-microbial agents. Other formulas are also possible, as described herein.

The dissolution time for an oral formulation can be modified for a rapid effect or for sustained release. Oral formulations may also contain a mixture of slow and fast release particles to produce rapid and sustained absorption in the same dose. Special coatings can be used with oral formulations such as tablets and capsules to impart resistance to stomach acids. Oral formulations can also be coated with sugar, varnish, or wax to improve taste.

Thus, tablets may be prepared as rapid dissolve tablets and capsules may be prepared as extended release capsules. The tablets may be scored tablets, chewable tablets, effervescent tablets, orally disintegrating tablets, or tablets for forming a suspension. The capsules may be gel capsules, for example, and may include powdered contents. This includes gel capsules made by single piece gel encapsulation and two piece gel encapsulation. Non-gelatine capsules are also included, as well as caplets.

It will be understood that certain formulations will be suitable for either antiseptic or pharmaceutical applications. Particular formulations of interest are: eye formulas (e.g., drops, ointments), car formulas (e.g., drops, ointments), nasal or airway formulas (e.g., drops, sprays, insufflation compositions, inhalation compositions, nebulisation compositions), skin formulas (e.g., soaps, sprays, aerosols, gels, pastes, lotions, creams, ointments, pads, patches, tapes, bandages, dressings, sponges, vapours) throat or mouth formulas (e.g., drops, lozenges, mouthwashes, toothpastes, sprays, pastes, gels), mucous membrane formulas (e.g., sprays, aerosols, gels, pastes, lotions, creams, ointments, pads, dressings, sponges).

Solid and liquid compositions can combine zingerone or a zingerone extract with one more compounds to ensure a stable and active composition. For example, an oral formulation, such as a tablet or capsule, may include: about 5 to about 50% w/w zingerone or zingerone extract; up to about 80% w/w of one or more fillers, lubricants, glidants, or binders; and up to about 10% w/w of compounds to ensure easy disintegration, disaggregation, and dissolution of the tablet in the stomach or the intestine.

Thus, the composition may contain various excipients, for example, one or more: solubilizers, stabilizers, buffers, tonicity modifiers, bulking agents, viscosity enhancers/reducers, surfactants, chelating agents, adjuvants, anti-adherents, anti-caking agents, binders, coatings, disintegrants, lubricants, glidants, flow agents, sorbents, flavours, colours, sweeteners, or preservatives. The composition may include less than 1% of a preservative, for example, about 0.005% to about 0.5%, or about 0.05% to about 0.15%, or may include about 0.04%, about 0.06%, about 0.08%, about 0.1%, about 0.12%, about 0.14%, about 0.16%, about 0.18%, or about 0.2% of a preservative, these percentages being representative of w/v values or w/w values. Useful preservatives include but are not limited to sorbic acid, sodium sorbate, potassium sorbate, citric acid, ascorbic acid, malic acid, tartaric acid, propionic acid, and benzoic acid, for example, in the form of its sodium salt, e.g., sodium benzoate.

Other useful excipients include but are not limited to: stearin, magnesium stearate, and stearic acid; saccharides and their derivatives, e.g., disaccharides: sucrose, lactose; polysaccharides and their derivatives, e.g., starches, cellulose or modified cellulose such as microcrystalline cellulose and cellulose ethers such as hydroxypropyl cellulose; sugar alcohols such as isomalt, xylitol, sorbitol and maltitol; proteins such as gelatin; synthetic polymers such as polyvinylpyrrolidone, polyethylene glycol; fatty acids, plant based surfactants; e.g., sunflower lecithin, waxes, shellac, plastics, and plant fibres, e.g., corn protein zein; hydroxypropyl methylcellulose; cross linked polymers, e.g., cross linked polyvinylpyrrolidone (crospovidone), and cross linked sodium carboxymethyl cellulose (croscarmellose sodium); sodium starch glycolate; silicon dioxide, fumed silica, talc, and magnesium carbonate.

Liquid compositions may be stored as tinctures in vials, bags, ampoules, cartridges, or prefilled syringes. The composition may also be transferred from a vial to a larger container and mixed with other materials. Dried or evaporated compositions may be stored in vials, cartridges, dual chamber syringes, or prefilled mixing systems. Before administration, a dry-form composition may be reconstituted as a liquid before being administered.

Exemplary unit dosages of the composition include: about 0.1 mg to about 1000 mg zingerone or zingerone extract, about 1 mg to about 500 mg zingerone or zingerone extract, about 1 mg to about 200 mg zingerone or zingerone extract, about 1 mg to about 100 mg zingerone or zingerone extract. The dosage may be formulated for administration once per week, twice per week, three times per week, every other day, once per day, twice per day, or three times per day, or more as needed. The dosage may be adjusted for paediatric, geriatric, overweight, underweight, or other patients, where required. Dosage modification can be made in accordance with known methods. The European Food Safety Authority (EFSA) has classified zingerone safe for human consumption with No Observed Adverse Effects (NOAEL) based on a dose of 128 mg/kg/day (EFSA 2016; 14 (8): 4557). It is to be appreciated therefore that a wide range of unit dose forms may be envisioned.

### Methods of Using the Compositions

As noted above, the disclosed compositions can be used to reduce the prevalence of microbial organisms and/or to treat or prevent various health conditions caused by such organisms.

In certain aspects, the composition may comprise (or may consist essentially of) zingerone or a zingerone extract, as produced by the methods set out herein. The composition of the present disclosure may also be formulated for one or more of: disinfection, antiseptic applications, or therapeutic applications. In addition, or as an alternative to this, the composition can be utilised as a functional food or beverage, a natural ingredient (e.g., a natural additive), or a natural supplement (e.g., a dietary supplement).

In various aspects, the disclosed compositions may be used to target one or more bacteria, fungi, or protozoan organisms. Specific fungi include but are not limited to: Aspergillus fumigatus, Aspergillus flavus, Candida albicans, Cryptococcus neoformans, Cryptococcus gattii, Histoplasma capsulatum, Pneumocystis jirovecii, Pneumocystis carinii, and Stachybotryschartarum. Of particular interest are drug resistant strains.

Specific protozoa include but are not limited to: Acanthamoeba culbertsoni, Acanthamoeba polyphaga, Acanthamoeba castellanii, Acanthamoeba astronyxis, Acanthamoeba hatchetti, Acanthamoeba griffini, Acanthamoeba lugdenensis, Acanthamoeba polyphaga, Acanthamoeba rhysodes, Balamuthia mandrillaris, Cryptosporidium parvum, Entamoeba histolytica, Giardia lamblia, Leishmania donovani, Leishmania infantum, Leishmania major, Naegleria fowleri, Plasmodium vivax, Plasmodium malariae, Plasmodium falciparum, Plasmodium ovale, Plasmodium knowlesi, Trypanosomacruzi, Trypanosoma brucei gambiense, Trypanosoma brucei rhodesiense, and Toxoplasma gondii. Of particular interest are drug resistant strains.

Specific bacteria include but are not limited to: Bacillus anthracis, Bacillus cereus, Bartonella henselae, Bartonella quintana, Bordetella pertussis, Borrelia burgdorferi. Borrelia garinii, Borrelia afzelii, Borrelia recurrentis, Brucella abortus, Brucellacanis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisellatularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasmaurealyticum, Vibrio cholerae. Yersinia pestis, Yersinia enterocolitica, and Yersinia pseudotuberculosis. Of particular interest are drug resistant strains, e.g., antibiotic resistant organisms.

In particular aspects, the disclosed compositions may be used together with one or more anti-microbial agents. For example, the composition may be prepared as a combined formulation with one or more anti-microbial agents. Alternatively, the composition may be utilised as a separate formulation along with one or more anti-microbial agents. Where separate formulations are used (e.g., a zingerone composition and an anti-microbial agent), it is possible to coordinate use by simultaneous or sequential application/administration of the separate formulas. In addition, a composition as described herein may be used in conjunction with various medical or non-medical procedures. Use of the composition may be carried out prior to, during, or after the procedure(s), or any combination thereof.

As examples, anti-microbial agents include but are not limited to: ethanol, isopropanol, glutaraldehyde, formaldehyde, triclocarbon, phenol, o-phenol, chlorophenol, amylmetacresol, thymol, cresol, resorcinol, chloroxylenol, triclosan, hexachlorophene, chlorhexidine, propamidine, hypochlorous acid, chloramine, iodophores, iodine, povidone iodine, dibromin, mercuric chloride, thiomersal, silver nitrate, silver sulfadiazine, zinc sulphate, zinc oxide, hydrogen peroxide, ozone, peracetic acid, cetrimide, benzalkonium chloride, gentian violet, basic fuchsine, methylene blue, acriflavine, salacrin, mercurochrome, boric acid, acetic acid, azelaic acid, nitrofuran, ethylene oxide.

Other anti-microbial agents include natural products, including essential oils or plant extracts, such as those from witch hazel, hops, thyme, oregano, calendula, tea tree, lavender, and anise, and specifically including lemon oil, orange oil, grapefruit oil, lime oil, neroli oil, mandarin oil, citronella oil, petitgrain oil, marjoram oil, rosemary oil, thyme oil, thymol, oregano oil, basil oil, clove oil, tea tree oil, juniper oil, myrrh oil, patchouli oil, pepper oil, black pepper oil, rose oil (e.g., rose otto oil), spikenard oil, vetiver oil, vervain oil, fennel oil, lemongrass oil, cinnamon oil, lavender oil, geranium oil, sandalwood oil, eucalyptus oil, pine oil, fir oil, balsam oil, cedar leaf oil, cedar wood oil, spearmint oil, wintergreen oil, peppermint oil, and menthol. Included also are honey (e.g., manuka honey), activated charcoal, yarrow (e.g., for various skin formulas), and comfrey (e.g., for ointments or creams).

Further included as anti-microbial agents are various antibiotics, for example, bacitracin, ceftriaxone, ciprofloxacin, clarithromycin, clindamycin, chloramphenicol, dapsone, dexamethasone, flucloxacillin, framycetin, fusidic acid, gentamicin, gramicidin, lincomycin, macrolide, mupirocin, nadifloxacin, neomycin, nitrofurazone, polymyxin B, retapamulin, soframycin, and sulfadiazine. Additional antibiotics include, for example, ampicillin, amoxicillin, e.g., amoxicillin with clavulanic acid, amoxicillin clavulanate, azithromycin, cefotaxime, cephalexin, ciprofloxacin, clioquinol, dicloxacillin, doxycycline, erythromycin, flumetasone, metronidazole, nafcillin, nitrofurantoin, ornidazole, oxacillin, penicillin, e.g., benzathinc penicillin, phenoxymethylpenicillin, penicillin G sodium, penicillin V potassium, roxithromycin, sulfamethoxazole, trimethoprim, and vancomycin. Noted amongst these are glycopeptide antibiotics such as dalbavancin, oritavancin, ramoplanin, teicoplanin, telavancin, and vancomycin. Noted in particular are aminoglycoside antibiotics such as gentamicin (e.g., Cidomycin®, Garamycin®, G-Myticin®, Pred-G®, Gentak®, Genoptic®), and also, amikacin, amikacin liposome, dibekacin, kanamycin, neomycin, netilmicin, paromomycin, plazomicin, sisomicin, streptomycin, and tobramycin. It will be understood that any combination of anti-microbial agents may be utilised in the methods and compositions of this disclosure.

As described herein, compositions of the present disclosure are useful as anti-microbial formulations. In particular aspects, antiseptic compositions can be used in methods to inhibit or halt microbial growth on or in certain tissue. This tissue includes: skin, nails, car, eye, nose, mouth, gums, throat, vaginal, and urinary tract tissues, and others noted herein. The antiseptic compositions may be applied, for example, to burns, to lessen the chance of infection, or to the skin before surgery, to combat microbial organisms on the skin around the operation site. The antiseptic compositions may be used as hand cleansers (e.g., soaps or hand sanitisers), to be applied with or without water. The antiseptic compositions may be used for minor skin infections, cuts, or grazes. The antiseptic compositions may be used as mouthwashes or gargles, for example, to combat microbial organisms in the mouth or on the gums. Antiseptic compositions may also be utilised as lozenges and throat sprays, for example, to relieve a sore throat. Antiseptic eye drops or ointments may be used to combat microbial organisms in or on the eye.

The compositions of the present disclosure also find use as formulations, which can be used in methods for treating or preventing microbial infections or other conditions resulting from infections, as described herein. The infection may affect one or more physiological components, including one or more parts of: the circulatory system, respiratory system, digestive system, renal system, excretory system, reproductive system, integumentary system, nervous system, lymphatic system, endocrine system, muscular system, skeletal system, and sensory systems.

Various routes of administration may be used for the compositions, including parenteral (e.g., topical) and enteral (e.g., oral) administration, as described herein. Enteral administration may be by duodenal tubing or gastric tubing, including nasogastric tubing, or other known means. Oral administration may be by tablets, capsules, sachets, drops, elixirs, linctuses, solutions, emulsions, suspensions, draughts, purees, pastes, pomaces, syrups, gels, jellies, tonics, or other known means. Topical administration may be by drops, sprays, ointments, soaps, pads, sponges, dressings, bandages, or various other means. Different modes of administration are known in the art and may be utilised by a skilled person. The compositions disclosed herein are not limited to a particular form for administration.

As exemplary dosages, the compositions may be administered to obtain about a 1 to 5000 mg dose of zingerone or zingerone extract for an average 70 kg human subject, or about 1 to 1500 mg dose of zingerone or zingerone extract, or about 5 mg to about 500 mg dose of zingerone or zingerone extract, or about 1 mg to about 500 mg dose of zingerone or zingerone extract, or about 1 mg to about 150 mg dose of zingerone or zingerone extract, or about 1 mg to about 100 mg dose of zingerone or zingerone extract, or ABOUT 1 Mg to about 50 mg dose of zingerone or zingerone extract, or about 1 mg to about 30 mg dose of zingerone or zingerone extract, or about 1 mg to about 20 mg dose of zingerone or zingerone extract, or about 1 mg to about 15 mg dose of zingerone or zingerone extract, or about 1 mg to about 10 mg dose of zingerone or zingerone extract, for an average 70 kg human subject. This range of dosages is particularly useful for a ginger component (e.g., zingerone or zingerone extract) that is dried and milled to a powder. The dosages as indicated above may be administered once per day, twice per day, three times per day, or less or more, as needed. Administration may be made with food, or before a meal. The appropriate dosage and dosage form will be readily determined by a person of skill in the art.

### Examples

The examples described herein are provided for the purpose of illustrating specific embodiments and are not intended to limit this disclosure in any way.

### Example 1: Preparation of Zingerone

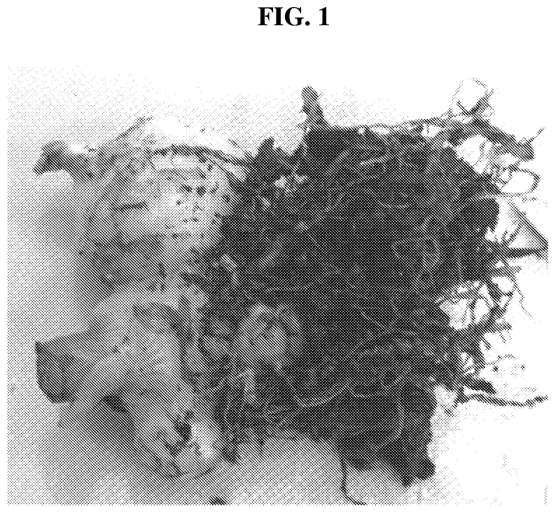

An initial sample of fresh ginger (400 gm) as shown in FIG. 1 was sourced from New Zealand and cleaned of dirt and soil. The cleaned ginger was then diced or chopped finely and subjected to alkaline treatment (800 g of 0.5% potassium hydroxide in distilled water). The resulting mixture was stirred and left in an oven at 60° C. for 22 hours. It is to be appreciated that the resulting mixture could also be placed in a water bath and maintained at around 60° C. for the desired time period.

The pH of the mixture was then adjusted to pH 7 by addition of concentrated citric acid and the treated plant material was spread out on a metal tray and dried in an oven at 60° C. for 20 hours. The resulting dried material weighed 35 grams resulting in a dry yield of 8.75%. The dried material was then scraped into a flask and covered with 95% ethanol for extraction (210 ml). The flask was shaken and placed in an oven at 40° C. for 16 hours. The extract (Extract 1) was filtered off using a glass funnel with a glass wool plug. The remaining plant material was extracted again with 95% ethanol (Extract 2) and then twice more with 50% ethanol (Extracts 3 and 4).

The extracts were analysed directly for zingerone content. A 294 mg chopped sample of the fresh ginger was also extracted with 2 ml of ethanol and this extract was also analysed. The results are shown in Table 1.

TABLE 1
Extract composition

| | | | Total | Total | *Dried |
|---|---|---|---|---|---|
| | Volume | Weight | zingerone | 6-gingerol | Weight |
| Extract | (mL) | (g) | (mg) | (mg) | (g) |
| 1 | 142 | 119 | 67 | 94 | 4.04 |
| 2 | 140 | 116 | 14 | 21 | 1.18 |
| 3 | 112 | 93 | 8 | 15 | |
| 4 | 104 | 86 | 3 | 9 | |
| Totals | | | 92 | 139 | |

*The dry weight of Extract 1 and Extract 2 was estimated by drying a 5 ml portion of each extract.

### Results

The 400 g fresh ginger root supplied was shown to contain 260 mg of 6-gingerol (i.e. 0.65 mg/g). This would mean the theoretical maximum yield of zingerone would be in the order of 171 mg from the treated material (loss of weight due to lower molecular weight of zingerone vs 6-gingerol). About 50% of the 6-gingerol was unconverted. It is envisioned that further studies could be used to increase the alkaline conversion of 6-gingerol to zingerone.

The ethanolic extraction was done using the minimal volume needed to cover the treated plant material. This meant that Extract 1, the most concentrated one, had a zingerone content of 0.47 mg/mL. It is envisioned that this concentration could, in principle, be increased using either a multi-steeping process or by evaporation of some of the ethanol. Reducing the volume of ethanol by 80% would yield a solution with 2.35 mg zingerone/mL.

It is envisioned that drying the extract fully would give a concentration of approximately 16 mg/g of solid extract. Higher concentrations would be expected if starting with a higher initial content of 6-gingerol and/or with more complete conversion. A theoretical 25 mg dose, for example, could then potentially be achieved by formulation of the dried extract directly into an oil or glycerol carrier to give the required zingerone dose in one or two 500 mg capsules.

The majority of the zingerone was extracted in the first extraction. There are situations where double extraction would be beneficial. The third and fourth extraction increased the totals by modest amounts. Overall, ethanol extraction was seen to be highly effective, and very efficient and inexpensive as a preparative method.

### Example 2: Preparation of Zingerone

Overview: These studies show that an aqueous alkaline treatment followed by freeze-drying of fresh ginger greatly improved conversion, reaching a ˜1% zingerone content in the dry ginger. The treated ginger was then extracted with supercritical CO₂ and CO₂+ethanol co-solvent with a combined extraction yield of 3% and an average concentration of zingerone in the extracted oleoresin of approximately 12%. In addition, it was found that drying of fresh ginger at a moderate temperature (60° C.) followed by supercritical CO₂ extraction resulted in an extraction yield of 4.6%. The extracted oleoresin was then alkaline treated and the final product contained approximately 15% zingerone.

Drying: Samples of the fresh ginger material imported from Fiji were sliced into 2-5 mm slices and placed on a single layer on perforated oven trays. Drying was performed in a forced convection. Drying was carried out at a moderate temperature (60° C.) with the goal of eliminating moisture without conversion of gingerol. This process was considered finished when the moisture content of the ginger reached 7%. The dried ginger obtained was stored refrigerated until it was used in the extraction trials.

Catalysed conversion: Small scale, preliminary trials were performed by treating around 1.6 g of fresh chopped Fijian sourced ginger with aqueous solutions of 0.5% KOH (pH 14), 1% Ca(OH)₂ (pH 11.6) and 1% sodium carbonate (pH 10.5). The volume:weight ratio of reagent added:ginger was around 3:1. The samples were then shaken and placed in a fumehood at room temperature, or ovens at 37° C. or 60° C., overnight, before being analysed. It is to be appreciated that suitable water baths could be used to keep the samples at the desired temperature for the desired period of time.

Once the alkaline treatment had been selected, 5.2 kg of fresh ginger was minced using a vertical cutter mixer (RobotCoupe R45). The ginger was then mixed with a 3:1 liquid:solid ratio (volume:weight) of KOH 0.5% (˜0.1 N), resulting in a pH˜ 12.5. The mixture was manually stirred and placed in an oven at 60° C. for 24 hours. After this time, the mixture was neutralised by adding concentrated citric acid (625 g/L) to a pH˜ 7.2. The neutralised mixture was then freeze-dried and the ginger obtained in this process was stored refrigerated until it was used in the extraction trials.

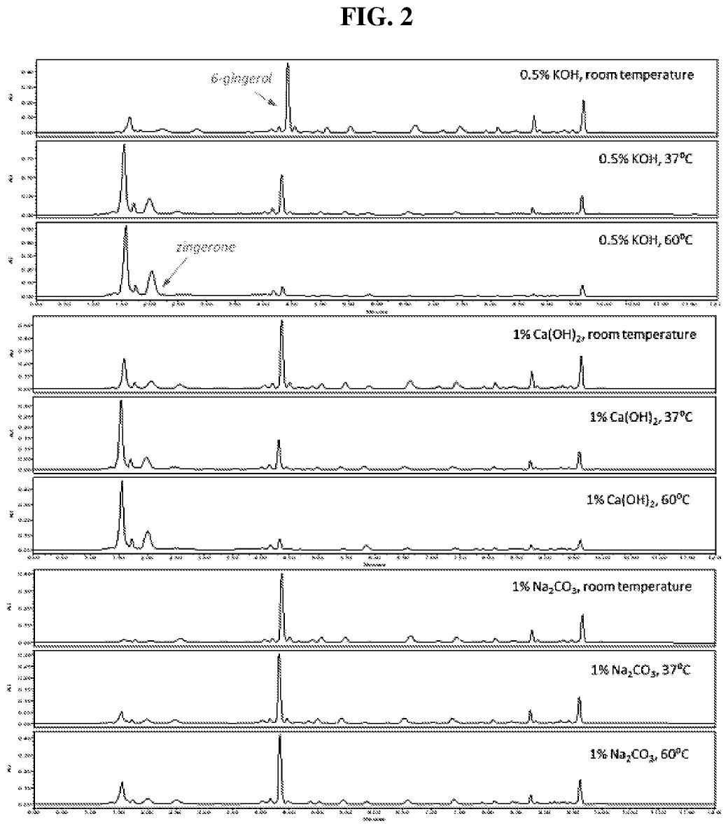

Catalysed conversion results: As noted, different alkalis were tested: 0.5% potassium hydroxide or KOH(pH 14), 1% calcium hydroxide or Ca(OH)₂ (pH 11.6), and 1% sodium carbonate or Na₂CO₃ (pH 10.5). The content of zingerone and 6-gingerol was quantified for these experiments (Table 2), and it was concluded that a treatment with KOH at 60° C. was the most efficient and gave the highest zingerone concentration. Table 2 shows the amounts of 6-gingerol mg/g and zingerone (mg/g) obtained for the respective conditions, with results expressed on a wet basis. HPLC traces are shown in FIG. 2. It can be seen from the HPLC traces that Ca(OH)₂ at 1% and KOH at 0.5% perform similarly. At 60° C. for KOH 0.5% the ratio of zingerone:6-gingerol is 6.5. At 60° C. for Ca(OH)₂ 1.0% the ratio of zingerone:6-gingerol is 4.0.

TABLE 2
Peak areas at 280 nm for zingerone
and 6-gingerol in treated samples

| Alkali | Temperature | 6-gingerol (mg/g) | Zingerone (mg/g) |
|---|---|---|---|
| KOH | Room temperature | 1.4 | 0.2 |
| KOH | 37° C. | 1.0 | 0.3 |
| KOH | 60° C. | 0.4 | 0.7 |
| Na₂CO₃ | Room temperature | 1.4 | 0.1 |

This treatment was applied to a larger sample of fresh ginger (5.2 kg) and the resulting treated ginger was then neutralised and freeze-dried. The yield of the freeze-dried ginger was 17%, i.e., 17 g of treated, freeze-dried ginger per 100 g of minced raw ginger. The zingerone and 6-gingerol content of the freeze-dried ginger was measured at 10.2 and 3.3 mg/g, respectively (dry basis). See Table 2-1. The zingerone content was at least 10 times higher than that of the oven dried ginger.

TABLE 2-1
Composition of treated and freeze-dried ginger

| | | Wet basis | Wet basis | Dry basis | Dry basis |
|---|---|---|---|---|---|
| | | 6-Gingerol | Zingerone | 6-Gingerol | Zingerone |
| Treatment | Moisture | (mg/g) | (mg/g) | (mg/g) | (mg/g) |
| KOH 0.5% | 4.5% | 3.2 | 9.8 | 3.3 | 10.2 |
| at 60° C. + | | | | | |
| freeze-dried | | | | | |

Extraction: Supercritical extraction trials were carried out using the alkaline treated ginger. The alkaline treated, freeze-dried ginger was lightly crushed by hand and placed in a 2 L extraction vessel with sintered filter discs at both ends, filling the vessel completely. The extraction was carried out as described above until a sharp decrease in the extraction rate was observed, corresponding with a CO₂:feed ratio of 13:1. At this point, the ethanol co-solvent pump was started and ethanol was added to the CO₂ stream with a ratio of approximately 10 wt % (i.e. 10 g ethanol per 100 g CO₂). The ethanol pump was stopped after a 2:1 ethanol:feed had been introduced (2 g ethanol per g of feed). CO₂ was then circulated to flush out any ethanol remaining in the bed. When the extraction was finished, the plant was depressurized, and the residual marc was allowed to degas overnight before being unloaded. The ethanol present in the extract was removed by rotary evaporation under vacuum. Extraction parameters are listed in Table 3.

TABLE 3
Summary for extraction parameters

| | Extraction 1 | | Extraction 2 | |
|---|---|---|---|---|
| Feed material | Untreated ginger | | Alkaline treated ginger | |
| | (dried at 60° C.) | | (freeze-dried) | |
| Solvent phase | CO₂ | | CO₂ => CO₂ + ethanol | |
| Extraction pressure | 300 | bar | 300 | bar |
| Extraction temperature | 40° | C. | 40° | C. |
| Separator pressure | 55 | bar | 55 | bar |
| Separator temperature | 40° | C. | 40° | C. |
| Feed mass | 358.6 | g | 322.41 | g |
| Average CO₂ flow rate | 2.8 | kg/h | 2.8 | kg/h |
| Average ethanol flow rate | | | 4.5 | g/min |

| | | |
|---|---|---|
| Ethanol % | | 10% |
| Ethanol to feed ratio | | 2:1 |
| CO₂ to feed ratio | 37.5:1 | 55:1 |

Analysis: Samples were prepared for analysis by addition of methanol after neutralisation as needed. Extracts were dissolved directly in methanol. Analysis was by HPLC with an acetonitrile/0.1% formic acid gradient. Detection was performed at 280 nm, and the column used was a Phenomenex Kinetex C18 (150×2.1 mm). Zingerone eluted at around 2 minutes and 6-gingerol at 5.2 minutes. Quantification of zingerone and 6-gingerol was obtained from a standard curve prepared using analytical standards of these compounds.

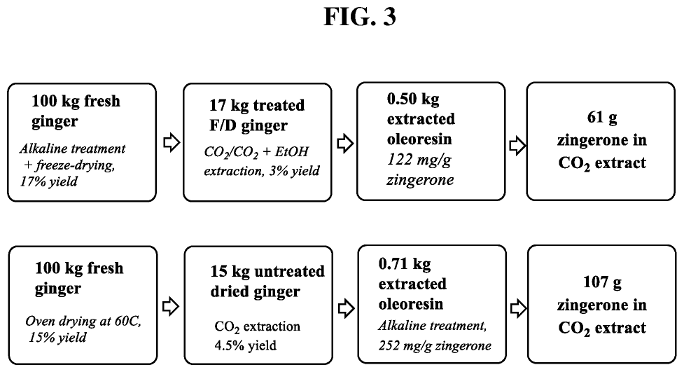

Extraction results: As noted, the alkaline treated and subsequently freeze-dried ginger was extracted with CO₂ followed by CO₂+ethanol co-solvent. No free water was observed in the CO₂ extract, and the ethanol obtained in the CO₂+ethanol extract was removed by rotary evaporation under vacuum. The extracts had a sweet, caramelized fragrance. The yield obtained with CO₂ was 1.5%. Addition of 10% ethanol co-solvent allowed for extraction of an additional 1.5%. The composition of the different fractions is shown in Table 4 and Table 5. See, also, FIG. 3.

TABLE 4
Composition of extracts obtained from alkali treated ginger

| | | 6-Gingerol | Zingerone |
|---|---|---|---|
| | | (mg/g) | (mg/g) |
| untreated | CO₂ extract | 290 | 0 |
| treated | CO₂ extract | 103 | 153 |
| treated | CO₂ extract + | 52 | 91 |
| | ethanol | | |
| treated | Marc | 1.2 | 5.4 |

TABLE 5
Zingerone (Z) mass balance for the extraction of alkali treated ginger

| | | | | | | CO₂ extract + | | | | | | |
|---|---|---|---|---|---|---|---|---|---|---|---|---|
| Feed | | | CO₂ extract | | | EtOH extract | | | Marc | | | Mass |
| g | Z mg/g | Z mg | g | Z mg/g | Z mg | g | Z mg/g | Z mg | g | Z mg/g | Z mg | balance |
| 322 | 9.8 | 3143 | 4.8 | 153 | 735 | 4.7 | 91 | 428 | 312 | 5.4 | 1687 | 90.6% |

It can be seen from the results that for the alkali treated sample, the CO₂ extract contained 153 mg/g of zingerone (15.3%) and 103 mg/g (10.3%) 6-gingerol, with a zingerone/gingerol ratio of approximately 1.5. The CO₂+ethanol extract contained 91 mg/g of zingerone and 52 mg/g of gingerol (zingerone/gingerol ratio of approximately 1.75). The marc or residual ginger post extraction was also analysed, and found to contain 5.4 mg/g of zingerone. When the mass of feed, extracts and marc is taken into account (Table 4), the zingerone mass balance can be calculated at 90.6%. This indicates that 90.6% of the zingerone present initially in the feed is accounted for in the extracts and marc. The difference could be caused by degradation during extraction or in the ethanol removal step. The zingerone extraction yield (i.e., grams of zingerone extracted per 100 g of zingerone in the feed) was only 37% when calculated based on the extracts. However, 54% of the initial zingerone present in the feed remains unextracted in the marc, so the zingerone extraction yield can also be calculated as 46% when based on the marc results. This accounts for the “missing” zingerone. Since the proportion of unextracted zingerone is significant, the extraction process could be further improved to reduce this. The extraction yield of gingerol is higher than that of zingerone (82% in the untreated sample and 71% in the treated sample), since it is more soluble in CO₂.

In another experiment, a CO₂ extract obtained from untreated ginger root was subjected to an alkaline treatment to study the conversion of gingerol into zingerone. Overnight treatment with both 0.1 N and 1 N KOH at 60° C. worked well, with a resulting concentration of zingerone in the treated ginger of around 15%. The resulting material appeared to be much cleaner than the alkaline treated crude ginger, so is an interesting alternative process that could provide an even more cost effective extraction process overall as the conversion process is on a smaller volume of material. In fact, for the same amount of starting fresh ginger (100 kg), and based on the results obtained in this work, the extraction of untreated ginger followed by alkaline treatment of the extract would yield almost twice as much zingerone in the final product than the alternative process (see comparison in the table below). However, even in this case, the overall zingerone yield of the process is ˜0.1% (0.1 kg zingerone per 100 kg fresh ginger). It is expected that further optimisation is possible.

In another experiment a sample of CO₂ extract of untreated ginger was mixed with KOH and converted to a zingerone rich extract. The post-treatment neutralisation step involved the separation of the zingerone as a zingerone enriched resin from the aqueous reaction mixture. 3 grams of oleoresin was taking in duplicate and 9 ml of 1 N KOH was added to the samples in a plastic vial. The samples were then shaken and left overnight in a 60 degrees oven. The treated samples were then neutralised by the addition of 10 ml of 1 N HCl. This was a small excess of acid to ensure that all of the KOH was neutralised. The addition was performed in two steps with mixing after each step. The samples were centrifuged at 2000 rpm to separate the water from the oleoresin. After centrifugation the bulk of the water was then removed by pipette. The resultant resin was then removed. Absolute alcohol was added to one of the resin samples (5 ml ethanol in approximately 3 grams resin) to produce a tincture (Sample 1). The other sample was kept in resin form (Sample 2). Sample 1 and Sample 2 were analysed for zingerone content. The concentration of zingerone in the tincture (Sample 1) was calculated at 23 mg/g of tincture while that of the treated resin (Sample 2) was 52 mg/g.

These values for both Samples 1 and 2 were lower than those obtained with earlier treatments for which 150 mg/g zingerone content was estimated. In this work the resin Sample 2 had been separated from the water and analysed. Subsequently the separated water was also analysed and estimated to contain another 50-55 mg (approximately 25%) of zingerone for each 3 g batch. Zingerone has been reported as having quite limited water solubility so this result was quite unexpected. Because it appears that a significant portion of the zingerone is staying in the water and not separating out with the resin, this method is contrasted to direct addition of alcohol to the crude neutralised product.

Because the water is able to extract some of the zingerone from the resin it may also be possible to produce a high yield of zingerone after treatment by drying down the total neutralised alkaline treatment product. Further process optimisation is to be undertaken to improve yields given the finding that after alkaline treatment zingerone appears to be more soluble in water than previously reported.

### Example 3: Further Reaction Methods and Comparisons

Overview: Zingerone is not naturally present in ginger but is a conversion product from gingerol through a treatment process. In these studies, a sample of alkaline treated and dried ginger was received from Samoa and extracted with ethanol using two different sets of extraction conditions: extraction at 40° C. for 20 h (Extract A) and at room temperature for 7 days (Extract B). The resulting zingerone concentration of the extracts was found to be 41.4 mg/g for Extract A and 43.8 mg/g for Extract B.

Extraction A: The treated ginger produced by SROS (Scientific Research Organisation of Samoa) was supplied in two separate plastic bags. The contents of both bags were combined, frozen at −80° C. and subsequently milled using a knife mill (Wiley) with a 2 mm mesh attached. The milled material (782.3 g) was then placed in a round bottom flask along with food grade ethanol with a ratio of approximately 5:1 by weight. The flask was then placed in a water bath at 40° C. and 5 rpm stirring overnight (total extraction time 20 h). After this time, the mixture was filtered under vacuum and the ethanol was removed by rotary evaporation under vacuum to produce 43.8 g of highly viscous, dark brown resin with a characteristic ginger aroma. Extraction yield was 5.6%. 10 g of this resin (Extract A) were taken out and stored refrigerated and under nitrogen flush for possible future bioassays.

Extraction B: The ginger was frozen and milled as described above. The milled material (785.9 g) was placed in a bucket along with food grade ethanol with a ratio of approximately 5:1 by weight. The ginger was macerated in ethanol at room temperature (22-29° C.) over 7 days. Samples were taken on days 1, 3 and 7. After 7 days, the mixture was filtered under vacuum and the ethanol was removed by rotary evaporation under vacuum to produce 49.3 g of highly viscous, dark brown resin with a characteristic ginger aroma (FIG. 1), very similar to the one obtained in Extraction A. 10 g of this resin (Extract B) were taken out and stored refrigerated and under nitrogen flush for possible future bioassays. Extraction yield was 6.3%.

Zingerone and aldehyde analysis: Quantification of zingerone was carried out by HPLC in the starting material (i.e., treated ginger) and the final two resins, as well as in the samples on days 1, 3 and 7 for Extraction B (note that the liquid sample from day 7 is equivalent to the final resin sample). The HPLC quantification method included methanol addition and grinding the sample before analysis. Zingerone content for all fractions is shown in Table 6. Zingerone mass balance and yield are shown in Table 7.

TABLE 6
Comparing extraction conditions - zingerone content for different fractions

| | Treated | Day 1 | Day 3 | Day 7 | Final |
|---|---|---|---|---|---|
| | ginger | sample | sample | sample | resin |
| Zingerone | 3.1 mg/g | | | | 41.4 mg/g |
| extract A | | | | | |
| Zingerone | 3.1 mg/g | 0.38 mg/ml | 0.44 mg/ml | 0.4 mg/ml | 43.8 mg/g |
| extract B | | | | | |

TABLE 7
Zingerone mass balance and yield

| | | Zingerone | Zingerone | | Zingerone | Zingerone | | |
|---|---|---|---|---|---|---|---|---|
| | Feed | in feed | in feed | Extract | in extract | in extract | Zingerone | Extraction |
| | (g) | (mg/g) | (g) | (g) | (mg/g) | (g) | yield | Yield |
| Ext A. | 782.3 | 3.1 | 2.4 | 43.8 | 41.4 | 1.8 | 75% | 5.6% |
| Ext B. | 785.9 | 3.1 | 2.4 | 49.3 | 43.8 | 2.2 | 89% | 6.3% |

As indicated above, Extract A was obtained by treatment at 40 degrees, duration 20 hrs, while Extract B was obtained by treatment at room temperature, duration 7 days. The results for Tables 6 and 7 show that higher levels of zingerone were obtained by longer treatment at room temperature, although high yields were also obtained by a temperature increase to 40° C.

To determine zingerone content in the starting material, this was first extracted into a suitable solvent. In one process, this extraction was carried out with ethanol resulting in a lower zingerone content (1.44 mg/g). In a second process, this was carried out using methanol and grinding the ginger along with the solvent in a mortar and pestle. This resulted in a higher zingerone content (3.1 mg/g). For reference, the zingerone content reported by the Samoan lab for this material was 1.86 mg/g.

Based on this, the zingerone yield (i.e., the amount of zingerone in the extract relative to the zingerone in the feed) was estimated at 75% for Extraction A and 89% for Extraction B. The 6-gingerol peak seen in the HPLC analysis when determining zingerone content was consistently observed at around ⅛th the peak area of zingerone. This suggests that extraction had little effect on the zingerone to gingerol ratio.

A sample of both final resins was taken up in ethanol and examined by GCMS for aldehyde analysis. A very low level of hexanal was seen (too low to quantify). Hexanal is a side product of the reaction to form zingerone but is somewhat volatile. The identity of the hexanal peak was confirmed by library matching of MS data and a separate injection of a hexanal standard.

### Discussion

Each of the studies described herein was effective in producing zingerone. A comparison of the results from Examples 2 and 3 is provided in Table 8.

TABLE 8
Comparison of zingerone yields

| | Zingerone | | Difference to fresh ginger |
|---|---|---|---|
| Fresh ginger | 0.1 | mg/g (wet basis) | Baseline |
| Dried ginger (180° C., 10 hr) | 0.7 | mg/g (wet basis) | 7-fold higher |
| Samoan ginger prototype | 43.8 | mg/g | 438-fold higher |
| (Example 3) | | | |
| Fijian ginger | 310 | mg/g expected | 3100-fold higher |
| (Example 2) | | | |
| Fijian ginger | 88.9 | mg/g | 889-fold higher |
| (Example 6) | | | |
| Fiji ginger | 177.8 | mg/g expected | 1778-fold higher |
| (Example 5) | | | |

Similar values for fresh and dried ginger were obtained by Li et al. See Li et al., 2016, “Chemical characterization and antioxidant activities comparison in fresh, dried, stir-frying and carbonized ginger” J Chromatogr B Analyt. Technol. Biomed. Life Sci. 1011:223-32. Example 2, as described above, utilises the retro-aldol reaction. Example 3, as described directly above, utilises temperature and pH adjustments and extraction.

Regarding the Samoan ginger, it is noted that the ginger was not harvested at the requested time (9 months in the ground) and this affected the level of gingerol present in the ginger and subsequently the zingerone contend in the end product. Therefore, it is expected that further gains can be obtained. Regarding the Fijian ginger, this was substantially higher in zingerone compared to the Samoan ginger (10.2:1.44=7.08× higher). This means that the total yield can be extrapolated for the Fijian ginger as 310 mg/g, if the experimental conditions of Example 3 were to be applied. That is: 43.8 mg/g (amount obtained from Samoan ginger in Example 3)×7.08 (higher starting content in Fijian ginger)=310 mg/g.

Table 5 in Example 2 shows the output of the pH treated ginger followed by CO₂ extraction. It was found that 322 grams of fresh ginger provides 153 mg/g zingerone. In comparison to this, Example 3 utilises 785.9 grams (2.4 times more compared to amount of product used in Example 2) and provides 43.8 mg/g zingerone. However, this lower yield can be explained by the lower levels in the starting material from Samoa (1.44 mg/g zingerone).

### Example 4: Processing Methods Using Juicing and Alkaline Treatment

Summary: Ginger root was mechanically juiced and the levels of 6-gingerol were determined for juice and remnant solids. The majority of the 6-gingerol was present in the juice. Treatment of the juice with alkali showed that effectively all of the 6-gingerol was converted to zingerone in 5-6 hrs at 60° C.

Overview for juicing: Fresh ginger (500 to 1000 g) was pre-treated by blending/macerating and pressing the ginger. The liquid fraction was retained and the marc was further washed with warm water (4 parts water to 1 part ginger) at a temperature of 55-60° C. for a period of 10-15 minutes. This was done in a covered vessel. This was then pressed again. Each fraction was analysed for 6-gingerol content (a total of 5 analyses), namely: 1) Fresh ginger sample immediately prior to processing; 2) Liquid fraction following initial blending/maceration; 3) Ginger marc following initial blending/maceration; 4) 2nd liquid fraction collected following further washing of initial marc; 5) Final ginger marc following washing then pressing as above. A moisture content reading was made on each lot of ginger marc, after final pressing (i.e. samples 3) and 5) above).

Overview for alkaline treatment: An alkaline treatment was performed on the liquid fractions to establish the conversion to zingerone. Samples were taken at a range of timepoints and analysed for zingerone content. The samples were subjected to small scale treatments with KOH (5% as previously). Treatment was carried out using 1 ml samples at room temperature, 30° C. and 60° C. samples at 1, 2, 3 and 5 hrs. Additionally, one sample was treated at 60° C. for 24 hours. Analysis of up to 15 samples was carried out.

Juicing method: Two samples of fresh ginger root were obtained, one locally (organic, from Evithe in Petone) and a second received from Phil Rasmussen in Auckland. Both samples appeared to be plumper/juicier than normal supermarket ginger root. The moisture content was determined by slicing up approximately 10 g of each root, freezing with liquid air and then freeze drying. The 6-gingerol content was determined extraction of root with methanol. For this, around 5 g of each sample was cut into 4-5 pieces and crushed using a small kitchen garlic press. The crushed root and juice were extracted with methanol (2×15 ml) at 60° C. for 20 minutes. This was followed by HPLC analysis with detection at 280 nm. The results are shown in Table 9, below.

TABLE 9
Moisture content of gingers

| Sample | % solid after freeze drying | 6-gingerol content (mg/g) |
|---|---|---|
| Auckland sample | 16 | 1.46 |
| Petone sample | 5.3 | 1.58 |

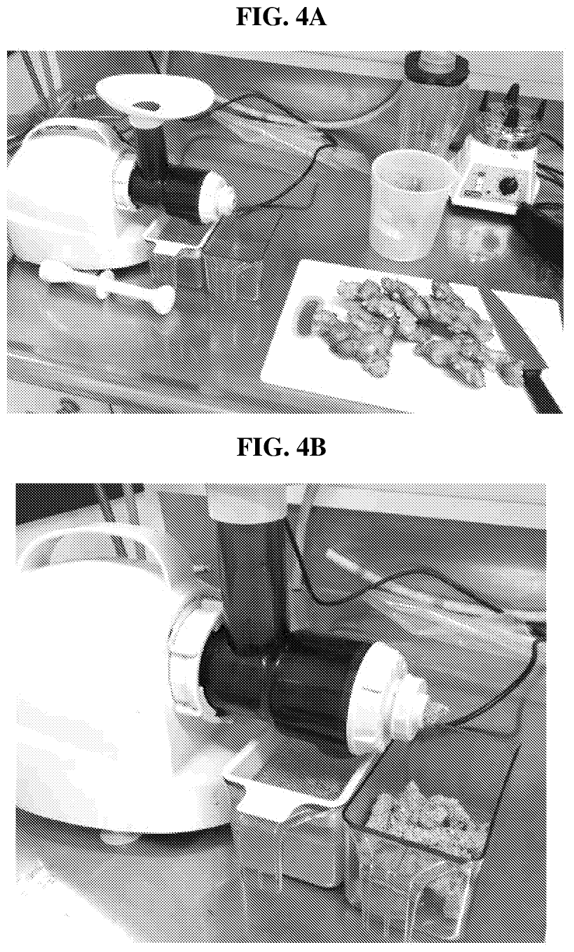

The Auckland sample was chosen for the juicing work. For this, 635 g ginger root was processed using a home juicer. This contained a rotating screw drive with mesh juice filter and adjustable solids nozzle (see FIGS. 4A-4B). The juicer removed 516.3 g (81%) of liquid (juice 1, J1) with 101.5 g of solid (marc 1, M1) collected. Some of the dry solid was removed for analysis and 90.5 g extracted with 360 ml of hot tap water. This was allowed to sit for 15 minutes before running this material through the juicer. From this step, 350 g of juice (juice 2, J2) was recovered along with 70.2 g of solid (marc 1, M2).

The two liquids were refrigerated overnight. Both were cloudy with settled solids. These samples were shaken up prior to analysis or treatment. The juice samples were analysed for 6-gingerol content by mixing a sample with ethanol (1:1), centrifuging and direct injection of the supernatant. The solid content of the two pressed solids M1 and M2 were 37% and 26.3% respectively.

The results are shown in Table 10, below. The gingerol values were multiplied by weight to give the total amounts of gingerol in each material. The juice was found to contain 81.6% of the measured gingerol. The total gingerol calculated for the feed was lower than the recovered amount suggesting a partial extraction of gingerol from the root. The percentage gingerol was based on the total measured gingerol for the marc and juices (rather than the feed measurement).

TABLE 10
Weights and gingerol content

| | weight | 6-gingerol | total | % |
|---|---|---|---|---|
| sample | (mg) | (mg/g) | 6-gingerol (mg) | 6-gingerol |
| Feed | 635 | 1.46 | 928 | |
| Juice 1 | 516.3 | 1.58 | 814 | 75 |
| Juice 2 | 350 | 0.2 | 72 | 6.6 |
| Marc 1 | 101.5 | 1.95 | 198 | 16.3 |
| Marc 2 | 90.5 | 1.816 | 146 | 13.4 |
| Sum of Juice 1, | | | 1085 | |
| 2 + Marc 2 | | | | |

Alkaline treatment: This work was performed on the first juice (J1) recovered from the juicing process (above). Samples of juice (after shaking to suspend all solids) were allowed to react with KOH at RT. 30 or 60° C. for 1, 2, 3, 5, and also at 60° C. for 24 hrs. Three concentrations of KOH were also trialled, 0.5, 1.0 and 2.0%. A solution of 2 N KOH was prepared (5.6 g KOH in 50 ml water). To generate 0.5%, 1% and 2% KOH concentrations in each sample, 0.25, 0.5 or 1 ml of the 2 N KOH was added to 5.5 ml of juice and shaken. The samples were then placed at RT (lab), 30° C. (water bath) or 60° C. (drying oven). Sampling for HPLC analysis was done by taking 200 μl from each sample, adding 200 μl of 1 N HCl, and then 500 μl of ethanol. After centrifugation the sample was directly injected into the HPLC. The peak areas of zingerone and gingerol were compared (see below).

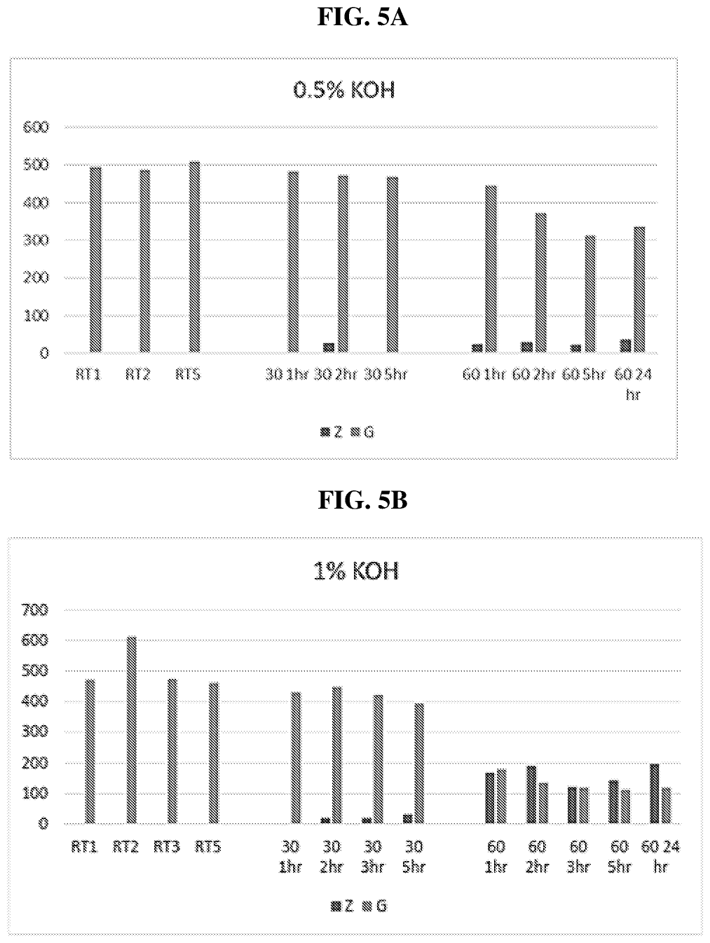

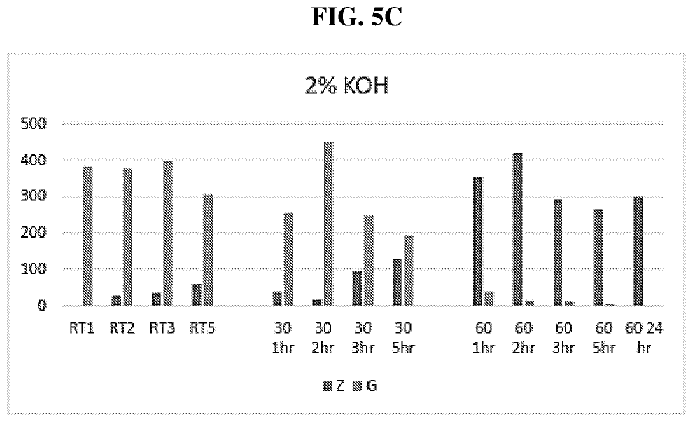

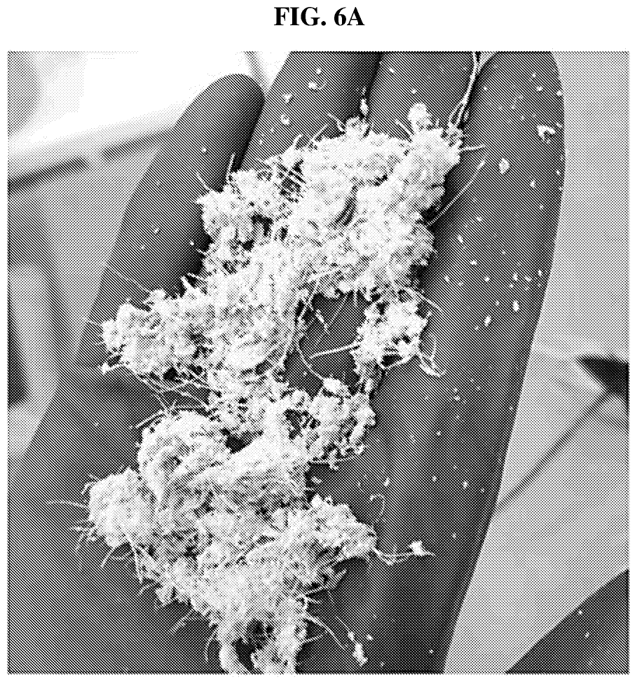

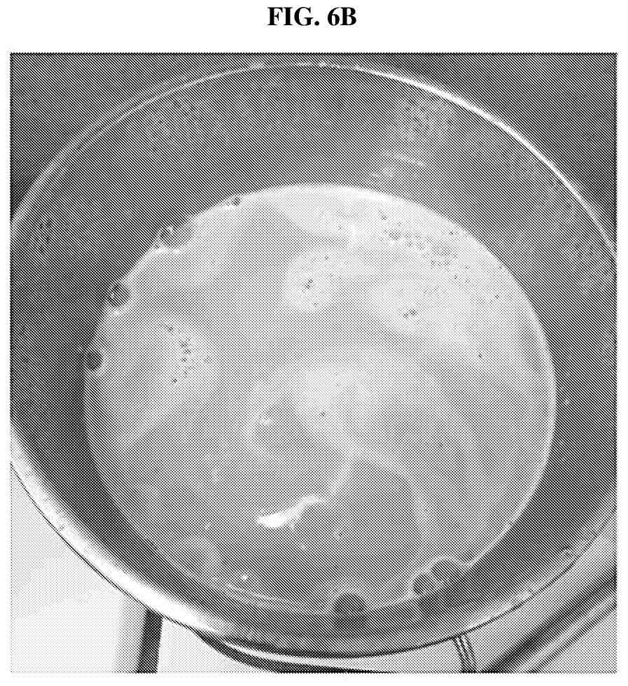

These results demonstrated that treatment with 2% KOH was able to achieve complete conversion to zingerone within 5 hours. See FIG. 5C. Incubation at 60° C. was particularly favourable. See FIG. 5C. The results are presented as peaks areas for zingerone (Z) and gingerol (G). It was noted that the KOH treated samples had solid present which settled in the tube. For analysis, the tubes were shaken and a sample was taken with a wide bore tip to avoid plugging.

### Example 5: Additional Processing Methods Using Juicing and Alkaline Treatment

Overview: The aim is to produce an extract from ginger having the 6-gingerol converted to zingerone by an alkaline-catalysed reversed aldol reaction. The current process seeks to reduce treatment time and water use. This process was trialled at a scale of roughly 40 kg before further production at a 200 kg scale will be undertaken.

In brief, fresh ginger was received and processed by treating it in alkali followed by freeze-drying to produce a treated ginger powder. This powder was extracted with ethanol at room temperature for 3 days, with samples taken at 24, 48 and 72 hours to assess the progress of the extraction. The ethanol extraction is detailed in Example 6.

Methodology: Alkaline pre-treatment and drying of imported fresh ginger was carried out. For this, fresh ginger (36.67 kg) was pressed in a Vincent Corporation CP-4 screw press to make two streams: a ginger juice and a marc. Screw Press settings were a VSD speed of 50%, and a cone air pressure of 2 bar. The marc from the initial press was pressed a second time to remove any remaining juice.

The two juices were combined. The juice was heated to 60° C. and KOH was added to a final concentration of 2% w/w. The alkalised juice was held at 60° C. for 5 hours to convert gingerol to zingerone. The juice was neutralised to a pH of 7.2 by addition of anhydrous citric acid. The juice, now containing zingerone, was freeze-dried and ground.

Samples were taken of: the fresh ginger (ZINGO); the two marcs (GMARC and GMARC2); the juice before KOH additions (GKOH0); the juice after 2, 3, and 5 hours of treatment (GKOH2, GKOH3, GKOH5); the final dried extract (GPE).

In addition, a sample of GMARC2 was extracted with hot water as follows. Water was added to GMARC2 at a ratio of 5:1 w:w. The mixture was heated to 60° C. and held at this temperature for 15 minutes. The extract was separated from the solids by screw pressing with the same settings as above. Samples were taken of: the extract (GMARC2 HWEX); the marc (HW MARC). Each sample was analysed for total solids (LOD, 16 hrs 100° C.) and gingerol or zingerone content by HPLC.

Sample analysis: Analysis was performed using HPLC with UV detection at 280 nm. Sample preparation was as follows: (1) liquid samples such as juice were diluted 1:1 with ethanol and centrifuged. In-process liquid samples with alkali present were diluted with 1 N HCl and ethanol 1:1:1 (2) solid samples such as raw ginger or ginger marc were extracted by double extraction with ethanol (ultrasonication, heat at 60° C. for 20 minutes, vortexed and centrifuged) and combining supernatants. Solid extracts (from approximately 5 g) were generally made up to 50 mL for analysis. The raw fresh ginger was roughly chopped and then blended with ethanol using an ULTRA-TURRAX® type mixer. Quantification was performed by comparison to a standard curve prepared using zingerone. A molecular weight correction was made for gingerol.

Pressing: 36.67 kg of raw Fijian ginger was received and pressed. Pressing was effective, producing a large volume of light green juice, and a fibrous marc. 28.92 kg of juice was recovered from first press. 6.6 kg of marc was then pressed a second time to recover an additional 2.18 kg of juice. Total yield of juice was 31.1 kg, equivalent to 86% of the incoming raw ginger by mass. The final marc mass recovered was 4.05 kg. There was typically 1-2 kg of holdup in the screw press at the end of a run. This gave rise to a minor difference noted between the feed mass and the combined mass of marc and juice.

Hot water extraction: 3.62 kg of GMARC2 and 18.1 kg of water were heated to 60° C. and then separated by screw pressing after 15 minutes of extraction at 60° C. Next, 21.18 kg of mixture was pressed. Noting that approximately 500 g of water was lost as evaporation during the extraction. From this, 17.62 kg of extract was recovered and subsampled for analysis. In addition, 2.69 kg of marc was recovered.

Alkaline treatment: For this, 1.236 kg of 50% KOH solution was added to the 31.1 kg of ginger juice, to reach a target KOH concentration of 2%. The pH after KOH addition was 12.18. On addition of KOH the colour of the juice changed for a light green to a reddish brown. The juice was held at 60° C. for 5 hours, and then neutralised by the addition of 500 g anhydrous citric acid. The pH after citric acid addition was 7.23.

Freeze drying: The treated juice was transferred to freeze dryer trays and frozen overnight before transferring to the Cuddon FD80 freeze dryer. A total of 28.51 kg of juice was loaded onto trays and dried. Approximately 3 kg of juice was lost prior to freezing and drying as a result of manual handling. After drying, 2.86 kg of dried extract was collected—a total of 10% of the mass of juice dried. This was ground and subsampled. After grinding and subsampling and handling losses a total of 2.19 kg was packed into foil bags for storage until further processing. Approximately 1.6 kg of this was sent for ethanol extraction (see Example 6).

Mass balance summaries are shown below.

### Juicing

| | | | |
|---|---|---|---|
| Inputs | fresh ginger | 100 | kg |
| Outputs | ginger juice | 85.79 | kg |
| | ginger marc | 14.21 | kg |

### Alkaline Treatment

| | | | |
|---|---|---|---|
| Inputs | ginger juice | 85.79 | kg |
| | 50% KOH solution | 3.37 | kg |
| | anhydrous citric acid | 1.36 | kg |
| Outputs | treated ginger juice | 90.53 | kg |

### Freeze Drying

| | | | |
|---|---|---|---|
| Inputs | treated ginger juice | 90.53 | kg |
| Outputs | dried ginger extract | 9.08 | kg |

Gingerol and zingerone levels were measured as described above. The results are shown in Table 11, below.

TABLE 11
Gingerol and zingerone levels

| | 6-Gingerol | Zingerone | |
|---|---|---|---|
| Sample | (mg/g) | (mg/g) | Basis |
| ZINGO (feed) | 5.22 | N/A | Wet |
| GMARC 1 | 1.08 | N/A | Wet |
| GMARC 2 | 1.99 | N/A | Wet |
| MARC HW | 0.22 | N/A | Wet |
| GKOH-0 | 6.90 | 0.00 | Dry |
| GKOH-2 | 0.93 | 5.76 | Dry |
| GKOH-3 | 0.90 | 5.74 | Dry |
| GKOH-5 | 0.79 | 5.32 | Dry |
| GPE | N/A | 5.70 | Dry |

This table shows the gingerol content of different fractions during pre-treatment and the treatment process. From these measurements, there was a total of 19.1 g of 6-gingerol in the 36.67 kg of raw ginger feed. The dried ginger extract (GPE) had a zingerone content of 5.7 mg/g. Therefore, the total zingerone in the 2.86 kg of dried powder (before losses and milling losses), was 16.30 g. The concentration of gingerol in the juice before conversion (GKOH-0) was 6.9 mg/g on a dry basis. When converted back to a wet basis, using a solids concentration of 6.67% (before the addition of KOH and anhydrous citric acid increased the total solids content to approximately 10%), the total gingerol in the juice was 14.32 g. The GKOH-2, -3, -5 values for 6-gingerol were to be confirmed.

The mass balances between the gingerol in the juice, and the zingerone in the final powdered extract are not precisely aligned. This may be due to variations in measurements. The marc numbers appear to be somewhat elevated, in view of the total mass balance calculations. When the marc was extracted with hot water, the extract had a 6-gingerol content of 0.22 mg/g, corresponding to a total of 5.7 g of 6-gingerol in this extract, roughly 25% of the 6-gingerol in the feed. For water extraction, this required a total of 18 kg of water. This in turn would increase the mass of KOH and citric acid, and the drying loads by an additional 58%. Thus, in certain circumstances, it may be desirable to omit water extraction.

Notably, these experiments showed that almost total recovery of 6-gingerol (in the form of zingerone) in the final product was achieved. The samples taken during the conversion reactions—GKOH2, GKOH3, and GKOH5-show that the conversion from 6-gingerol to zingerone occurred during the first two hours of treatment, with no significant increase in zingerone levels in samples taken after 2 hours of treatment. Given that the conversion appears to be complete after 2 hours, this incubation time (or even shorter incubation times) will be sufficient.

It was concluded that the processing method, including pressing followed by KOH treatment in the juice phase, was an effective production method. Further experiments will employ more than 200 kg ginger as starting material. 6-Gingerol levels in this batch were 1.1 mg/g and this was expected to reflect in the corresponding zingerone levels.

### Example 6: Ethanol Extraction Process and Analysis

Overview: Fresh ginger was received and processed by treating it in alkali followed by freeze-drying to produce a treated ginger powder (see Example 5, above). This powder was extracted with ethanol at room temperature for 3 days, with samples taken at 24, 48 and 72 hours to assess the progress of the extraction.

Methodology: The treated ginger produced as above (Example 5) was stored refrigerated until used. Approximately half of the received ginger was extracted with XNS food grade ethanol at room temperature. The treated ginger, along with ethanol (using a ginger:ethanol ratio of 1:5 by weight), was placed in a 10 L glass vessel equipped with an overhead stainless-steel stirrer. The mixture was stirred for 72 hours, with a stirring speed sufficient as to not allow sedimentation of solids at the bottom.

After 24 hours had passed, the stirrer was turned off and the solids were allowed to settle for 10 minutes before taking a 50 mL sample from the top. After another 24 hours had passed, a second sample was taken out using the same procedure. After a total 72 hours had passed, stirring was stopped and the mixture was filtered under vacuum using filter paper. Samples were taken of the cake and a final tincture sample, representing 72 hours, was taken from the filtrate. The remaining filtrate was labelled ZINGOEE. A ˜300 mL sample of ZINGOEE was taken and stored refrigerated in a glass bottle. In a further step, all ZINGOEE was evaporated to create a total resin volume of 43.5 g at a value of 88.9 mg/g zingerone.

The remaining ethanolic tincture was evaporated under vacuum using a Buchi R220SE rotary evaporator operating at 50 mbar and 40° C. until a volume reduction of approximately 31-fold was achieved. The resulting concentrated extract (ZINGOCE) was then analysed and once the zingerone content was confirmed, a small sample of this concentrated extract was used to produce a standardised tincture containing ˜12 mg zingerone per gram by diluting it with food grade ethanol. Two separate samples of this standardised tincture were sent to SCU (Australia) for analysis, and a third sample was retained onsite for zingerone analysis.

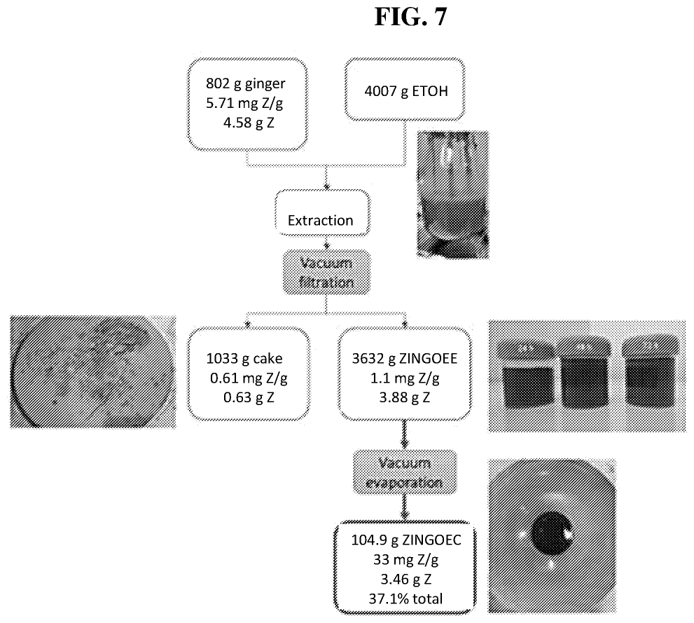

Results: The process and results are set out in FIG. 7. For these experiments, 801.5 g of the received treated ginger was used in the ethanol extraction, along with 4007.3 g of food grade ethanol. After 72 hours at room temperature (16-20° C.) under stirring, the mixture was filtered and 3556.7 g of a clear brown, aromatic ethanol tincture was obtained [ZINGOEE], as well as 1033.4 g of cake (i.e., spent ginger solids). Approximately 140 g of ethanol was lost due to evaporation during the extraction. The total weight of ethanolic tincture produced, including the samples taken at 24 and 48 hours, was 3631.7 g.

Quantification of zingerone was carried out by HPLC in the starting material (i.e., treated ginger, GPE) and the samples after 24, 48 and 72 hour ethanol extraction at room temperature. The spent ginger solids (i.e., the cake from the filtration process) was also analysed. Zingerone content for all fractions is shown in Table 12. HPLC results showed little difference between the three extraction times, indicating that 24 hours is sufficient for extraction.

TABLE 12
Zingerone content (mg/g) for samples

| Treated | | | | |
|---|---|---|---|---|
| ginger | 24 h | 48 h | 72 h sample | Spent ginger solids |
| [GPE] | sample | sample | [ZINGOEE] | [CAKE] |
| 5.7 | 1.07 | 1.08 | 1.1 | 0.6 |

The filtrate [ZINGOEE] had a zingerone concentration of 1.1 mg/g, i.e., 3.88 g zingerone are present in the liquid, or 85% of the starting zingerone, indicating reasonable recovery. The cake had a zingerone concentration of 0.61 mg/g, i.e., 0.63 g of zingerone in the cake. However, it was noted that the cake had certain amount of ethanolic solution still present. In future processing, the cake could be washed with clean ethanol to flush out as much extract as possible.

The zingerone mass balance for the ethanol extraction process is 98.5%. This is based on 3.88 g in the extract, plus 0.63 g in the cake, divided by 4.58 g in the feed. It was determined that room temperature extraction provided advantageous recovery of zingerone. It is possible that increasing the extraction temperature could lead to higher zingerone recovery, but room temperature extraction is clearly effective.

After taking ˜300 mL sample of ZINGOEE, the remaining extract (3249 g) was evaporated under vacuum to produce 104.9 g of concentrated extract [ZINGOCE] with a zingerone content of 33 mg/g (3.46 g of zingerone). This concentrated extract had a total solids content of 37.1% (measured as loss on drying at 110° C.), indicating that the final oleoresin weight that could be achieved if all ethanol was removed would be 38.9 g. If this number is extrapolated to the total amount of ZINGOEE produced, the resulting extraction yield for the process is around 5.4%. A standardised tincture containing 12 mg/g of zingerone was prepared by combining 25.5 g of ZINGOCE with 44.5 g of food grade ethanol. Samples of this tincture were sent for further testing.

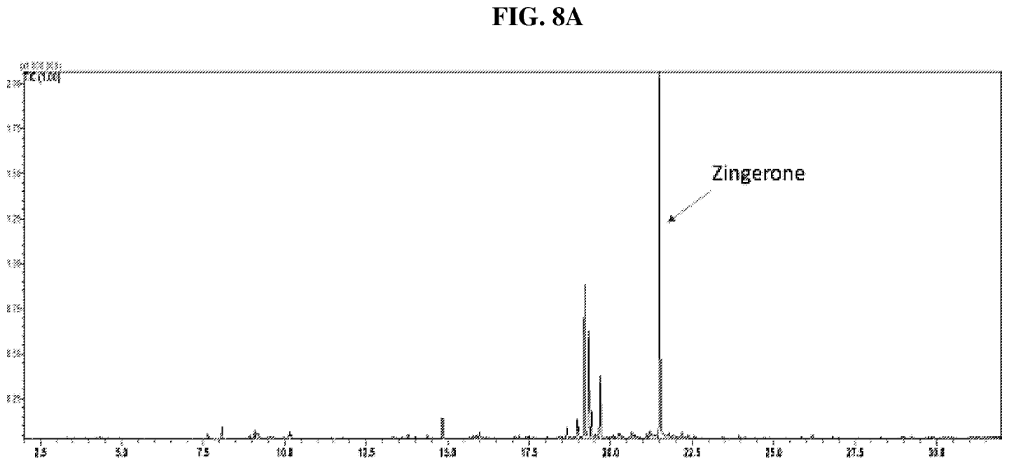

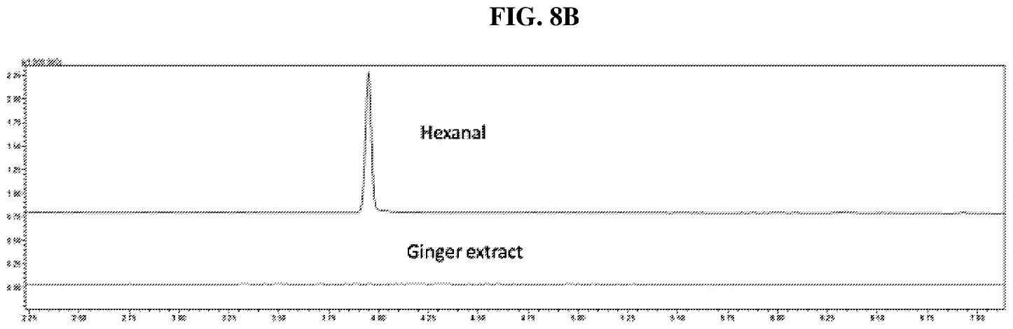

In addition, further analysis showed that no aldehydes were present in the final product. See FIGS. 8A-8B. For these assessments, a sample of an ethanolic extract of treated ginger was analysed using GCMS for the presence of aldehydes. If present, this would be expected as (predominantly) hexanal derived from 6-gingerol. It is expected that the powder preparations (e.g., pre-extraction powders, as in Examples 4 and 5, above) will also lack aldehydes. Next steps will include evaporation of the ethanolic tincture to produce a thick ethanolic paste.

Conclusions: The proposed method which included screw pressing followed by KOH treatment in the juice phase, neutralisation with citric acid, and then freeze drying, proved to be highly effective, with almost total recovery of 6-gingerol in the juice phase achieved, as well as total conversion to zingerone after 5 hours of treatment with 2% KOH at 60° C. The pre-processed dried powder currently contained 5.32 mg/g zingerone, which is at least two times more efficient than previous manufacturing techniques.

The ethanol extraction at room temperature for 24 hours is an optional step achieving at least 85% recovery of zingerone. The recovery could be further increased by washing the solids with fresh ethanol after extraction. In these methods, the ethanolic extract [ZINGOEE] was evaporated to achieve a significant volume reduction, followed by reconstitution of the concentrated extract [ZINGOCE] with fresh ethanol in order to produce a standardised tincture containing the target dose of 12 mg/g zingerone.

For the extraction process, the overall Z mass balance (out/in) was 98.5%. The zingerone content in the filtrate (ZINGOEE) was 1.1 mg/g, and the zingerone content in the concentrated extract (ZINGOCE) was 33 mg/g. The method produced 104.9 g of ZINGOCE at 33 mg/g, leaving 3.46 g zingerone. This number is lower than the 3.88 g in ZINGOCE because ˜400 g of ZINGOCE was removed before evaporation (for testing, plus ˜300 mL retention sample). Taking this into account, the calculations fit very well. The overall zingerone mass balance before evaporation was calculated as 98.5% (=(0.63+3.88)/4.58).

The results obtained in this work indicate that 100 kg of fresh ginger with a 6-gingerol content of 0.5 mg/g would yield 3.6 kg of standardised tincture with a zingerone content of 12 mg/g.

### Example 7: Combinations with Zingerone and Anti-Microbial Agents

Gentamicin is an antibiotic in the class of aminoglycosides and is used to treat severe gram-negative bacterial infections. It is available in a range of dosage forms including injectable solution, as well as intravenous and oral formulations. Invasive Staphylococcus aureus bacteria often develop resistance against antibiotic mono-therapy with gentamicin. Several strains of Staphylococcus aureus develop resistance to gentamicin and other aminoglycosides via the expression of antibiotic modifying enzymes that mediate gentamicin bacterial resistance.

Vancomycin is an antibiotic in the class of glycopeptides and is the most widely used glycopeptide antibiotic for treating gram-positive infections in adults, children, and neonates. It is available in various formulations, including oral (e.g., capsule or solution), and intravenous injection. However, vancomycin-resistant enterococci have emerged, making treatment more difficult. E. faecium is the most common strain to acquire vancomycin resistance. The main mechanism of glycopeptide resistance (e.g., vancomycin) in enterococci involves the alteration of the peptidoglycan synthesis pathway, specifically the substitution of D-Alanine-D-Alanine (D-Ala-D-Ala), to either D-Alanine-D-Lactate (D-Ala-D-Lac) or D-Alanine-D-Serine (D-Ala-D-Ser).

Chequerboard analysis was used to determine whether compounds could be combined to decrease the MIC for Staphylococcus aureus, as compared to each individual compound. In the present experiments, combinations of zingerone plus gentamicin, zingerone plus vancomycin, and zingerone plus cefotaxime were tested. For this testing, 96 well plates were inoculated Staphylococcus aureus, and then incubated with the test compounds for 24 hours at 37° C. For zingerone, additions were made at 0, 0.78, 1.56, 3.125, 6.26, 12.5, and 25 mg/ml. For gentamicin, additions were made at 0, 4, 8, 16, 32, 64, 128, and 256 μg/ml. For vancomycin, additions were made at 0, 4, 8, 16, 32, 64, 128, and 256 μg/ml. For cefotaxime, additions were made at 0, 8, 16, 32, 64, 128, 256, and 512 μg/ml. MRSA cells (methicillin-resistant Staphylococcus aureus) were introduced in Mueller Hinton media with 2% NaCl. 10⁸ cells per ml were utilised. The FICI index was used to quantify the interaction of the compounds interact employing the following equation: FICI=(MIC_{A}^{combi}/MIC_{A}^{alone})+ (MIC_{B}^{combi}/MIC_{B}^{alone}). A FICI of ≤0.5 was designated as synergy, and a FICI of >4.0 was defined as antagonism. A FICI of 0.5 to 1.0 was deemed as a significant improvement although not synergistic. The results are set out in Tables 13-16, below.

TABLE 13
Minimum inhibitory concentration for individual compounds
used against Staphylococcus aureus bacteria.

| Compound | MIC | |
|---|---|---|
| Gentamicin | 16 | μg/mL |
| Zingerone | 25 | mg/g |
| Vancomycin | 8 | μg/mL |
| Cefotaxime | >512 | μg/mL |

TABLE 14
Combination of gentamicin and zingerone reduces
MIC against Staphylococcus aureus bacteria

| Compound | MIC | |
|---|---|---|
| Gentamicin | 4 | μg/mL |
| Zingerone | 6.25 | mg/g |

TABLE 15
Combination of vancomycin and zingerone reduces
MIC against Staphylococcus aureus bacteria

| Compound | MIC | |
|---|---|---|
| Vancomycin | 4 | μg/mL |
| Zingerone | 12.5 | mg/g |

TABLE 16
Combination of cefotaxime and zingerone as used
against Staphylococcus aureus bacteria

| Compound | MIC | |
|---|---|---|
| Cefotaxime | 512 | μg/mL |
| Zingerone | 25 | mg/g |

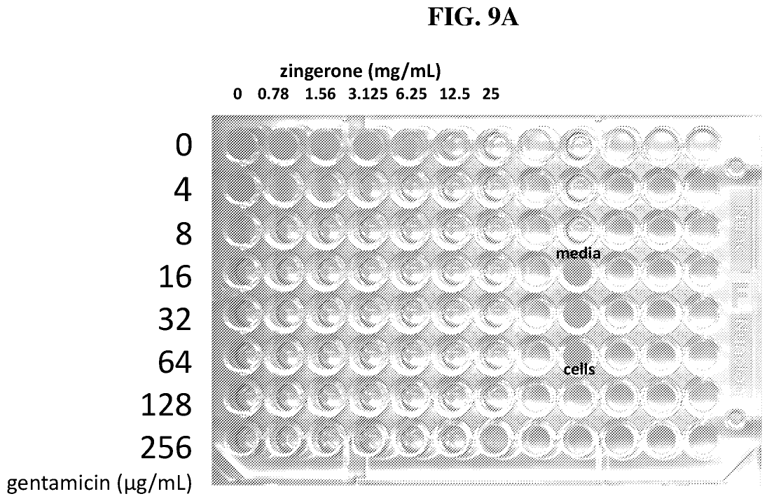

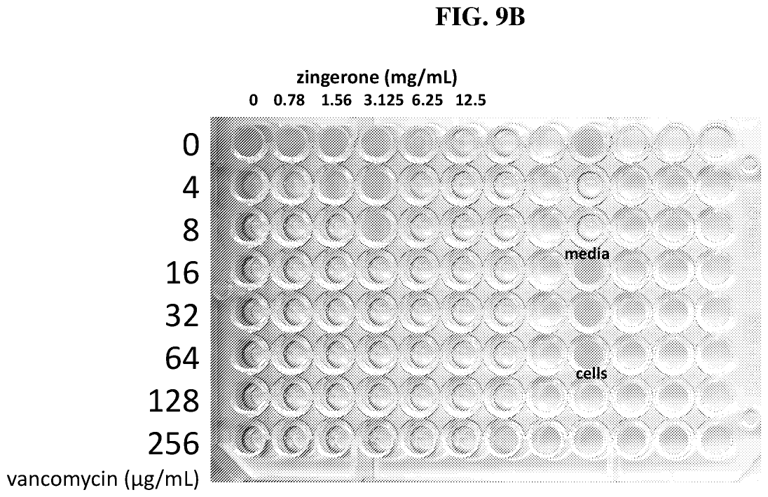

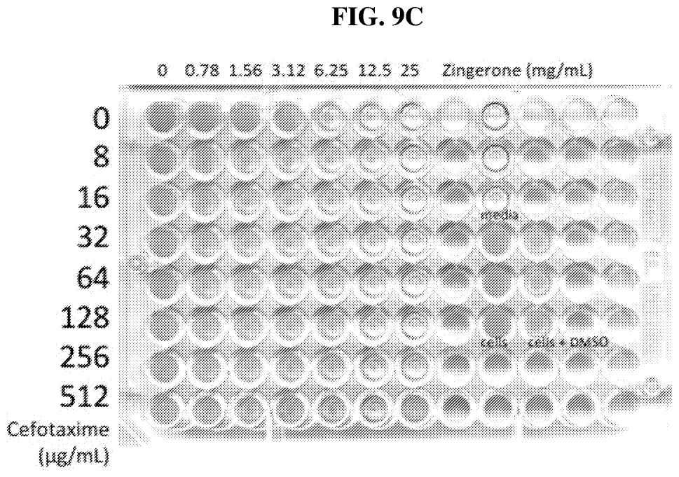

From these results, it was determined that gentamicin combined zingerone and vancomycin combined with zingerone reduced the MIC for Staphylococcus aureus as compared to gentamicin, vancomycin, and zingerone added individually (see FIGS. 9A-9B). For the combination of zingerone and gentamicin, this activity was synergistic: FICI=0.5=(MIC_{A}^{combi} 4 μg/mL/MIC_{A}^{alone} 16 μg/mL)+ (MIC_{B}^{combi} 6.25 mg/g/MIC_{B}^{alone} 25 mg/g). For the combination of zingerone and vancomycin, the inhibitory activity was significantly increased, and nearing synergistic activity: FICI=0.1=(MIC_{A}^{combi} 4 μg/mL/MIC_{A}^{alone} 8 μg/mL)+ (MIC_{B}^{combi} 12.5 mg/g/MIC_{B}^{alone} 25 mg/g). By comparison, the combination of zingerone and cefotaxime (which belongs to the cephalosporin family of antibiotics) did not produce any notable change in activity when compared to each compound applied individually.

Gentamicin and vancomycin are representative of aminoglycoside antibiotics and glycopeptide antibiotics, respectively. The advantageous increase in inhibitory activity for combinations of zingerone and gentamicin (synergistic activity), and zingerone and vancomycin (significant increase/near synergy) provides a valuable tool for use in fighting infection, particularly for fighting antibiotic resistant strains, including antibiotic resistant strains of Staphylococcus aureus. Such combination treatments provide increased potency and decreased likelihood of resistance, and are extremely beneficial as treatment modalities.

Persons of ordinary skill can utilise the disclosures and teachings herein to produce other embodiments and variations without undue experimentation. All such embodiments and variations are considered to be part of this disclosure.

Accordingly, one of ordinary skill in the art will readily appreciate from the present disclosure that later modifications, substitutions, and/or variations performing substantially the same function or achieving substantially the same result as embodiments described herein may be utilised according to such related embodiments. Thus, the present disclosure is intended to encompass, within its scope, the modifications, substitutions, and variations to processes, manufactures, compositions of matter, compounds, means, methods, and/or steps disclosed herein.

The description herein may contain subject matter that falls outside of the scope of the claimed invention. This subject matter is included to aid understanding of the invention.

In this specification, where reference has been made to external sources of information, including patent specifications and other documents, this is generally for the purpose of providing a context for discussing the features of this disclosure. Unless stated otherwise, reference to such sources of information is not to be construed, in any jurisdiction, as an admission that such sources of information are prior art or form part of the common general knowledge in the art.

1. A method of producing zingerone, comprising:
(i) subjecting ginger root to an alkaline treatment in an alkaline solution; or
(ii) subjecting juice obtained from ginger root to an alkaline treatment in an alkaline solution;
thereby producing zingerone.

2. The method as claimed in claim 1 wherein the ginger root is fresh.

3. The method as claimed in claim 1 or claim 2, wherein:
(a) the ginger root of (i) is chopped;
(b) the ginger root of (i) is chopped and dried; or
(c) the juice of (ii) is obtained via maceration and/or pressing.

4. The method as claimed in any one of claims 1 to 3, wherein:
(a) potassium hydroxide (KOH) is used in the alkaline solution; or
(b) a liquid form of potassium hydroxide (KOH) is used in the alkaline solution.

5. The method as claimed in claim 4, wherein the alkaline solution comprises:
(a) about 0.1% to about 1.0% KOH (v/v);
(b) about 0.5% to about 0.7% KOH (v/v);
(c) about 1% to about 3% KOH (v/v);
(d) about 1.5% to about 2.5% KOH (v/v); or
(e) about 2% KOH (v/v).

6. The method as claimed in any one of claims 1 to 3, wherein calcium hydroxide (Ca(OH)₂) is used in the alkaline solution.

7. The method as claimed in claim 6, wherein the alkaline solution comprises:
(a) about 0.5% to about 4% Ca(OH)₂ (v/v);
(b) about 1.5% to about 3.5% Ca(OH)₂ (v/v); or
(b) about 2.0% to about 3.0% Ca(OH)₂ (v/v).

8. The method as claimed in any one of claims 1 to 7, wherein:
(a) the alkaline treatment is carried out at about 40 to about 70 degrees Celsius;
(b) the alkaline treatment is carried out at about 50 to about 60 degrees Celsius;
(c) the alkaline treatment is carried out at about 55 to about 65 degrees Celsius; or
(d) the alkaline treatment is carried out at about 60 degrees Celsius.

9. The method as claimed in any one of claims 1 to 8, wherein:
(a) the alkaline treatment is carried out for about 1-30 hours, about 1-20 hours, about 1-10 hours, or about 1-5 hours;
(b) the alkaline treatment is carried out for about 0.5 to about 3 hours, or about 0.75 to about 2.5 hours, or about 1 to about 2 hours; or
(c) the alkaline treatment is carried out for about 1 hour, or about 2 hours.

10. The method as claimed in any one of claims 1 to 9, including the further step of:
(a) neutralising the alkaline solution; and/or
(b) extracting the zingerone following the alkaline treatment.

11. The method as claimed in claim 10, wherein the zingerone is extracted by:
(a) one or more ethanol extraction steps;
(b) supercritical fluid extraction; or
(c) a supercritical fluid extraction followed by an ethanol extraction step.

12. The method as claimed in any one of claims 1 to 11, wherein the method produces a product consisting essentially of zingerone.

13. The method as claimed in any one of claims 1 to 12, wherein the method produces a product that is free of aldehydes or substantially free of aldehydes.

14. A composition comprising zingerone, wherein the zingerone is obtained by the method of any one of claims 1 to 13.

15. The composition as claimed in claim 14, wherein the composition is formulated as a pharmaceutical composition.

16. The composition as claimed in claim 14 or claim 15, wherein the composition is formulated as a liquid or a powder.

17. The composition as claimed in any one of claims 14 to 16, which is formulated for topical administration or oral administration.

18. The composition as claimed in any one of claims 14 to 17, which is formulated as a liquid, powder, tablet or capsule.

19. The composition as claimed in claim 18, wherein the liquid, powder, tablet or capsule comprises:
(a) a dose of between about 1 mg to about 5000 mg of zingerone;
(b) a dose of between about 1 mg to about 1500 mg of zingerone;
(c) a dose of between about 5 mg to about 500 mg of zingerone;
(d) a dose of about 1 mg to about 15 mg of zingerone; or
(e) a dose of about 1 mg to about 10 mg of zingerone.

20. The composition as claimed in any one of claims 14 to 19, which is:
(a) formulated for co-administration with a further anti-microbial agent;
(b) formulated for co-administration with one or more aminoglycoside antibiotic agents or one or more glycopeptide antibiotic agents;
(c) formulated for co-administration with gentamicin or vancomycin;
(d) comprising gentamicin or vancomycin.

21. The composition as claimed in any one of claims 14 to 20 for use in treating or preventing an infection by a microbial organism.

22. The composition as claimed in claim 21, wherein the microbial organism is selected from the group consisting of: bacteria and fungi.

23. The composition as claimed in claim 22, wherein the bacteria are Gram positive bacteria or Gram negative bacteria.

24. The composition as claimed in claim 22, wherein the bacteria are selected from the group consisting of: Bacillus, Clostridium, Escherichia, Mycoplasma, Neissaria, Pseudomonas, Salmonella, Shigella, Streptococcus, Staphylococcus, and Vibrio bacteria.

25. The composition as claimed in claim 22, wherein the bacteria are selected from the group consisting of: Escherichia coli, Staphylococcus aureus, Streptococcus pneumoniae, Pseudomonas aeruginosa, and Klebsiella pneumoniae bacteria.

26. The composition as claimed in claim 22, wherein the fungi are selected from the group consisting of: Aspergillus, Candida, Coccidioides, Cryptococcus, Histoplasma, Pneumocystis, and Stachybotrys fungi.

27. The composition as claimed in claim 22, wherein the fungi are selected from the group consisting of: Candida albicans, Aspergillus species, Histoplasma capsulatum, Coccidioides immitis, and Pneumocystis carinii, and tinea fungi.

28. The composition as claimed in any one of claims 21 to 27, wherein the microbial infection is an infection affecting one or more of: skin, eye, ear, nose, mouth, throat, oesophagus, lung, circulatory system, gastrointestinal system, or genitourinary system.

29. Use of a composition as claimed in claim 14 for preparing a medicament for treating or preventing an infection by a microbial agent.

30. The use as claimed in claim 29, wherein the medicament provides for reducing or slowing progression of the infection.

31. The use as claimed in claim 29 or claim 30, wherein:
(a) the medicament is formulated as a liquid or a powder; and/or
(b) the medicament is formulated for topical administration or oral administration.

32. The use as claimed in any one of claims 29 to 31, wherein the medicament is formulated as a liquid, tablet or capsule.

33. The use as claimed in claim 32, wherein the liquid, tablet or capsule comprises:
(a) a dose of between about 1 mg to about 5000 mg of zingerone;
(b) a dose of between about 1 mg to about 1500 mg of zingerone;
(c) a dose of between about 5 mg to about 500 mg of zingerone;
(d) a dose of about 1 mg to about 15 mg of zingerone; or
(e) a dose of about 1 mg to about 10 mg of zingerone.

34. The use as claimed in any one of claims 29 to 33, wherein:
(a) the composition is formulated for co-administration with one or more anti-microbial agents;
(b) the composition is formulated for co-administration with one or more aminoglycoside antibiotic agents or one or more glycopeptide antibiotic agents;
(c) the composition formulated for co-administration with gentamicin or vancomycin;
(d) the medicament comprises gentamicin or vancomycin.

35. The use as claimed in any one of claims 29 to 34, wherein the microbial organism is selected from the group consisting of: bacteria and fungi.

36. The use as claimed in claim 35, wherein the bacteria are Gram positive bacteria or Gram negative bacteria.

37. The use as claimed in claim 35, wherein the bacteria are selected from the group consisting of: Bacillus, Clostridium, Escherichia, Mycoplasma, Neissaria, Pseudomonas, Salmonella, Shigella, Streptococcus, Staphylococcus, and Vibrio bacteria.

38. The use as claimed in claim 35, wherein the bacteria are selected from the group consisting of: Escherichia coli, Staphylococcus aureus, Streptococcus pneumoniae, Pseudomonas aeruginosa, and Klebsiella pneumoniae bacteria.

39. The use as claimed in claim 35, wherein the fungi are selected from the group consisting of: Aspergillus, Candida, Coccidioides, Cryptococcus, Histoplasma, Pneumocystis, and Stachybotrys fungi.

40. The use as claimed in claim 35, wherein the fungi are selected from the group consisting of: Candida albicans, Aspergillus species, Histoplasma capsulatum, Coccidioides immitis, and Pneumocystis carinii, and tinea fungi.

41. The use as claimed in any one of claims 29 to 40, wherein the microbial infection is an infection affecting one or more of: skin, eye, ear, nose, mouth, throat, oesophagus, lung, circulatory system, gastrointestinal system, or genitourinary system.

42. A method of treating or preventing an infection by a microbial agent, the method comprising administering to a subject a composition as claimed in claim 15, thereby treating or preventing the infection by the microbial agent.

43. The method as claimed in claim 42, wherein the administration reduces or slows progression of the infection.

44. The method as claimed in claim 42 or claim 43, wherein:
(a) the composition is administered as a liquid or a powder; and/or
(b) the composition is administered by topical administration or oral administration.

45. The method as claimed in any one of claims 42 to 44, wherein the composition is administered as a liquid, tablet or capsule.

46. The method as claimed in claim 45, wherein the composition is administered at:
(a) a dose of between about 1 mg to about 5000 mg of zingerone;
(b) a dose of between about 1 mg to about 1500 mg of zingerone;
(c) a dose of between about 5 mg to about 500 mg of zingerone;
(d) a dose of about 1 mg to about 15 mg of zingerone; or
(e) a dose of about 1 mg to about 10 mg of zingerone.

47. The method as claimed in any one of claims 42 to 46, wherein:
(a) the composition is co-administered with one or more anti-microbial agents;
(b) the composition is co-administered with one or more aminoglycoside antibiotic agents or one or more glycopeptide antibiotic agents;
(c) the composition is co-administered with gentamicin or vancomycin;
(d) the composition is formulated to include gentamicin or vancomycin.

48. The method as claimed in any one of claims 42 to 47, wherein the microbial organism is selected from the group consisting of: bacteria and fungi.

49. The method as claimed in claim 48, wherein the bacteria are Gram positive bacteria or Gram negative bacteria.

50. The method as claimed in claim 48, wherein the bacteria are selected from the group consisting of: Bacillus, Clostridium, Escherichia, Mycoplasma, Neissaria, Pseudomonas, Salmonella, Shigella, Streptococcus, Staphylococcus, and Vibrio bacteria.

51. The method as claimed in claim 48, wherein the bacteria are selected from the group consisting of: Escherichia coli, Staphylococcus aureus, Streptococcus pneumoniae, Pseudomonas aeruginosa, and Klebsiella pneumoniae bacteria.

52. The method as claimed in claim 48, wherein the fungi are selected from the group consisting of: Aspergillus, Candida, Coccidioides, Cryptococcus, Histoplasma, Pneumocystis, and Stachybotrys fungi.

53. The method as claimed in claim 48, wherein the fungi are selected from the group consisting of: Candida albicans, Aspergillus species, Histoplasma capsulatum, Coccidioides immitis, and Pneumocystis carinii, and tinea fungi.

54. The method as claimed in any one of claims 42 to 53, wherein the microbial infection is an infection affecting one or more of: skin, eye, ear, nose, mouth, throat, oesophagus, lung, circulatory system, gastrointestinal system, or genitourinary system.
